(19) United States
(12) Patent Application Publication
(10) Pub. No.: US 2025/0333607 A1
(43) Pub. Date: Oct. 30, 2025

(54) PROCESS FOR FABRICATING A REDUCED GRAPHENE OXIDE-BASED ANTIFOULING MARINE COATING MATERIAL

(71) Applicant: PRINCESS NOURAH BINT ABDULRAHMAN UNIVERSITY, Riyadh (SA)

(72) Inventors: Anuj KUMAR, Mathura (IN); Thamraa ALSHAHRANI, Riyadh (SA); Mohd UBAIDULLAH, Riyadh (SA); Hari Mohan SHARMA, Mathura (IN); Akshay PARMAR, Mathura (IN); Mohd SHKIR, Abha (SA); Yufeng ZHO, Shanghai (CN); Akshi TYAGI, Bihar (IN)

(73) Assignee: PRINCESS NOURAH BINT ABDULRAHMAN UNIVERSITY, Riyadh (SA)

(21) Appl. No.: 19/264,682

(22) Filed: Jul. 9, 2025

(51) Int. Cl. C09D 5/16; A61L 2/232; A61L 101/30; A61L 101/46; C01B 32/196; C01B 32/198; C08K 3/04; C08K 9/06; C09D 5/14; C09D 7/20; C09D 7/40; C09D 7/62; C09D 7/80; C09D 163/00

(52) U.S. Cl. CPC C09D 5/1618; A61L 2/232; C01B 32/196; C01B 32/198; C09D 5/14; C09D 7/20; C09D 7/62; C09D 7/67; C09D 7/80; C09D 163/00; A61L 2101/30; A61L 2101/46; C08K 3/041; C08K 3/042; C08K 9/06; C08K 2201/011; C08K 2201/014

(57) ABSTRACT

The present invention generally relates to a process for fabricating a reduced graphene oxide (rGO)-based antifouling marine coating is disclosed. The process begins with preparing a dispersed graphene oxide solution, followed by chemical reduction using hydrazine hydrate at 90-95° C. for 3 hours to form a dispersed rGO solution. This solution is then washed, filtered, and sonicated for 6 hours to ensure stability. A polymer solution is prepared by dissolving 40-50 wt % epoxy resin in acetone at 50° C. The antifouling composite is then formed by combining the rGO solution, 10-15 wt % zinc oxide nanoparticles, and 1-5 wt % carbon nanotubes with the polymer solution, followed by 6 hours of sonication at room temperature. Finally, the resulting composite material is applied to a substrate as a coating using spraying, brushing, dipping, or spin coating. This coating offers a promising solution for preventing biofouling on marine structures.

### FIELD OF THE INVENTION

The present disclosure relates to antifouling coatings for marine environments, and more particularly to a process for fabricating a reduced graphene oxide-based antifouling marine coating material that provides improved long-term antifouling performance due to the specific method of incorporating the reduced graphene oxide into the coating matrix.

### BACKGROUND OF THE INVENTION

Marine biofouling presents a considerable difficulty, resulting in elevated fuel consumption, structural impairment, and augmented maintenance expenses for boats and marine infrastructures. The aggregation of organisms like algae, barnacles, and mussels on submerged surfaces diminishes the efficiency of marine vessels by augmenting drag and increases material degradation, resulting in frequent repairs and replacements. This biofouling also impacts the structural integrity of fixed marine installations like offshore platforms and buoys.

Conventional antifouling coatings predominantly utilize hazardous biocides, such as tributyltin (TBT), which have been prohibited or limited in numerous areas due to their harmful impacts on marine ecosystems. These impacts include toxicity to non-target organisms and disruption of aquatic food webs. The environmental risks associated with these traditional antifouling solutions, coupled with increasing regulatory pressures, underscore the pressing necessity for sustainable, high-performance antifouling materials capable of mitigating biofouling while preserving environmental integrity.

Reduced graphene oxide (rGO), an innovative two-dimensional material composed of a single sheet of carbon atoms in a hexagonal arrangement, presents a sophisticated alternative to conventional antifouling techniques. Its remarkable mechanical strength guarantees endurance against physical degradation, while its thermal conductivity and chemical stability render it resistant to severe marine conditions, including ultraviolet light, salinity, and temperature variations. The intrinsic hydrophobic properties of rGO inhibit water infiltration, hence diminishing the likelihood of bio-organism adhesion. Furthermore, rGO's capacity for functionalization with diverse additives facilitates the integration of ecologically benign biocidal agents, thereby improving its antifouling efficacy while maintaining ecological compatibility.

While rGO holds significant promise, its effective incorporation into a durable and functional marine coating presents challenges. Simple mixing of rGO into a standard coating matrix often results in poor dispersion, weak adhesion to the substrate, and limited long-term performance. Therefore, there remains a need for a robust and scalable process for fabricating rGO-based antifouling marine coatings that address these limitations.

This invention utilizes the multifunctional characteristics of rGO alongside meticulously chosen polymer matrices and functional additives to develop an innovative, environmentally sustainable marine covering. The resultant material has exceptional antifouling features, improved adhesion, self-cleaning characteristics, and prolonged stability. This unique technology aims to transform the maritime industry by enhancing performance and sustainability, thereby lowering operational costs, decreasing environmental impact, and assuring adherence to progressively rigorous regulatory standards.

In view of the foregoing discussion, it is portrayed that there is a need to have a process for fabricating a reduced graphene oxide-based antifouling marine coating material.

### SUMMARY OF THE INVENTION

The present disclosure seeks to provide a process for fabricating a reduced graphene oxide-based antifouling marine coating material. This invention pertains to the creation and production of a reduced graphene oxide (rGO)-based antifouling marine coating material intended to inhibit biofouling on marine structures and boats. Biofouling, characterized by the growth of algae, barnacles, and other marine organisms on submerged surfaces, presents considerable challenges by augmenting drag, diminishing fuel efficiency, and hastening material degradation. The innovative coating formulation incorporates improved rGO or rGO-oxide derivatives, which provide superior mechanical strength, hydrophobicity, and chemical stability, alongside biocidal chemicals and polymer matrices to produce a high-performance solution. These biocidal chemicals are meticulously chosen to deliver efficient antifouling effects while reducing environmental damage, responding to increasing apprehensions regarding the ecological consequences of conventional antifouling coatings.

Besides its antifouling features, the material demonstrates self-cleaning characteristics that prevent the adherence of pollutants, hence minimising the necessity for regular cleaning and upkeep. The polymer matrix improves the coating's endurance, guaranteeing its integrity under severe marine conditions, including UV radiation, saltwater corrosion, and mechanical stress. The coating's superior adhesive capabilities enable it to attach securely with various substrates, such as steel, aluminium, and fibreglass, rendering it flexible for application in diverse marine infrastructure. Moreover, the extended durability of the coating substantially reduces the necessity for reapplication, resulting in cost efficiencies in maintenance and operating interruptions.

This approach provides a revolutionary answer for marine businesses by integrating advanced materials science with sustainable engineering practices. It not only improves the efficiency and durability of vessels and underwater structures but also aids global initiatives to minimise the environmental impact of marine operations.

In an embodiment, an antifouling marine coating material composition, comprising: 40-50% by weight of epoxy resin; 20-25% by weight of dispersed reduced graphene oxide; 10-15% by weight of zinc oxide nanoparticles; 1-5% by weight of carbon nanotubes; and 1-5% by weight of organic solvent.

In another embodiment, a process for fabricating a reduced graphene oxide-based antifouling marine coating material is disclosed. The process includes preparing a dispersed graphene oxide solution.

The process further includes preparing a dispersed reduced graphene oxide solution, by adding 30% w/v of a hydrazine hydrate solution to the dispersed graphene oxide solution, heating the resulting mixture to a temperature between 90° C. and 95° C. for approximately 3 hours with constant stirring, washing the resulting slurry with de-ionized water until the pH reaches a neutral point, filtering the washed slurry, immersing the filtered slurry in de-ionized wate, and sonicating the immersed slurry for approximately 6 hours to form a dispersed reduced graphene oxide solution.

The process further includes preparing a polymer solution by dissolving 40-50% by weight epoxy resin in acetone at a temperature of approximately 50° C. with stirring.

The process further includes forming a composite material by adding a suspension of 20-25% by weight of dispersed reduced graphene oxide solution to the polymer solution, adding 10-15% by weight of zinc oxide nanoparticles to the mixture, adding 1-5% by weight of carbon nanotubes, and sonicating the resulting mixture for approximately 6 hours at room temperature to form the composite material.

The process further includes applying the resulting composite material on a substrate as a coating by one of the spraying, brushing, dipping, or spin coating.

In one embodiment, the graphene oxide solution dispersion comprising combining 10 grams of graphene oxide paste with 50 mL of de-ionized water, and sonicating the resulting mixture for approximately 2 hours to form a dispersed graphene oxide solution.

In a further embodiment, the sonication is performed using a sonicator at a power and frequency sufficient to disperse the graphene oxide, wherein the sonication is performed at room temperature, wherein the graphene oxide paste comprises graphene oxide flakes and water.

Yet, in another embodiment, the mixture is stirred at 1000-2000 rpm before or during sonication.

Yet, in a further embodiment, the hydrazine hydrate solution is added dropwise to the dispersed graphene oxide solution, wherein the pH is considered neutral when it is within the range of 6.5 to 7.5.

Yet, one of the above embodiments, the sonication is performed at a temperature of approximately 25° C., wherein in the dispersed reduced graphene oxide is stored at 25° C.

In another embodiment, the epoxy resin is dissolved in acetone with stirring at approximately 1500 rpm at a temperature of approximately 50° C. for approximately 2 hours, wherein the acetone is pre-heated before adding the epoxy resin.

Yet, in another embodiment, preferably a Bisphenol A-type epoxy resin in added in the fabrication of antifouling marine coating material.

The process further comprising curing the applied composite material at room temperature for 1-3 days to form a solid, adhesive layer.

An object of the present disclosure is to develop a process for fabricating such an rGO-based antifouling marine coating material that is scalable and cost-effective.

Another object of the present disclosure is to create a marine coating that combines the mechanical strength, thermal stability, and hydrophobic properties of graphene with eco-friendly antifouling mechanisms, minimizing the reliance on harmful biocides.

Yet another object of the present invention is to deliver an expeditious and cost-effective reduced graphene oxide (rGO)-based antifouling marine coating material that exhibits superior resistance to biofouling, corrosion, and mechanical wear.

To further clarify the advantages and features of the present disclosure, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1 illustrates a flow chart of a process for fabricating a reduced graphene oxide-based antifouling marine coating material in accordance with an embodiment of the present disclosure;

FIG. 2 illustrates a curve of variation in contact angle as a function of rGO content in accordance with an embodiment of the present disclosure;

FIG. 3 illustrates a curve of coating loss (%) with the time in salty solution in accordance with an embodiment of the present disclosure;

FIG. 4 illustrates a curve of coating loss (%) with the time in humidity in accordance with an embodiment of the present disclosure;

FIG. 5 illustrates a variation in corrosion as a function of rGO content (%) in salty solution in accordance with an embodiment of the present disclosure;

FIG. 6 illustrates a curve of variation in contact angle as a function of rGO content in acrylic varnish in accordance with an embodiment of the present disclosure;

FIG. 7 illustrates a curve of variation in contact angle as a function of rGO content in artistic gloss varnish in accordance with an embodiment of the present disclosure; and

FIG. 8 illustrates a table depicting the experimental results data of the present invention.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail concerning the accompanying drawings.

In an embodiment, an antifouling marine coating material composition, comprising: 40-50% by weight of epoxy resin; 20-25% by weight of dispersed reduced graphene oxide; 10-15% by weight of zinc oxide nanoparticles; 1-5% by weight of carbon nanotubes; and 1-5% by weight of organic solvent.

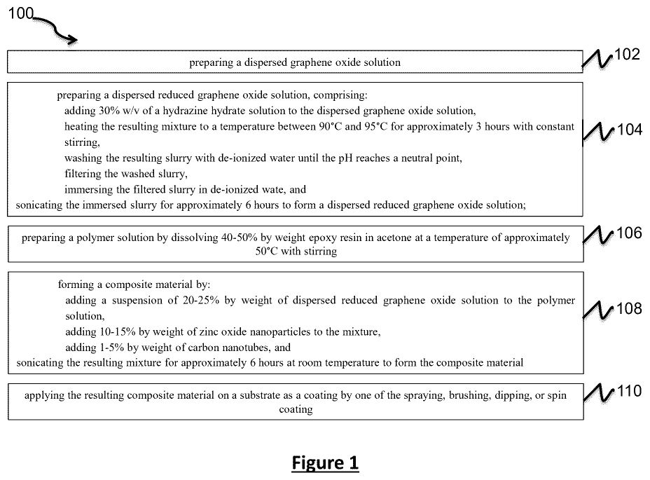

Referring to FIG. 1, a flow chart of a process for fabricating a reduced graphene oxide-based antifouling marine coating material is illustrated in accordance with an embodiment of the present disclosure. At step (102), process (100) includes preparing a dispersed graphene oxide solution.

At step (104), process (100) includes preparing a dispersed reduced graphene oxide solution, by adding 30% w/v of a hydrazine hydrate solution to the dispersed graphene oxide solution, heating the resulting mixture to a temperature between 90° C. and 95° C. for approximately 3 hours with constant stirring, washing the resulting slurry with de-ionized water until the pH reaches a neutral point, filtering the washed slurry, immersing the filtered slurry in de-ionized wate, and sonicating the immersed slurry for approximately 6 hours to form a dispersed reduced graphene oxide solution.

At step (106), process (100) includes preparing a polymer solution by dissolving 40-50% by weight epoxy resin in acetone at a temperature of approximately 50° C. with stirring.

At step (108), process (100) includes forming a composite material by adding a suspension of 20-25% by weight of dispersed reduced graphene oxide solution to the polymer solution, adding 10-15% by weight of zinc oxide nanoparticles to the mixture, adding 1-5% by weight of carbon nanotubes, and sonicating the resulting mixture for approximately 6 hours at room temperature to form the composite material.

At step (110), process (100) includes applying the resulting composite material on a substrate as a coating by one of the spraying, brushing, dipping, or spin coating.

In one embodiment, the graphene oxide solution dispersion comprising combining 10 grams of graphene oxide paste with 50 mL of de-ionized water, and sonicating the resulting mixture for approximately 2 hours to form a dispersed graphene oxide solution.

In a further embodiment, the sonication is performed using a sonicator at a power and frequency sufficient to disperse the graphene oxide, wherein the sonication is performed at room temperature, wherein the graphene oxide paste comprises graphene oxide flakes and water.

Yet, in another embodiment, the mixture is stirred at 1000-2000 rpm before or during sonication.

Yet, in a further embodiment, the hydrazine hydrate solution is added dropwise to the dispersed graphene oxide solution, wherein the pH is considered neutral when it is within the range of 6.5 to 7.5.

Yet, one of the above embodiments, the sonication is performed at a temperature of approximately 25° C., wherein in the dispersed reduced graphene oxide is stored at 25° C.

In another embodiment, the epoxy resin is dissolved in acetone with stirring at approximately 1500 rpm at a temperature of approximately 50° C. for approximately 2 hours, wherein the acetone is pre-heated before adding the epoxy resin.

Yet, in another embodiment, preferably a Bisphenol A-type epoxy resin in added in the fabrication of antifouling marine coating material.

The process further comprising curing the applied composite material at room temperature for 1-3 days to form a solid, adhesive layer.

In an embodiment, during the dropwise addition of the 30% w/v hydrazine hydrate solution to the dispersed graphene oxide, the solution is magnetically stirred at a constant speed of 1200 rpm while maintaining the pH in the range of 9.5 to 10.5 using intermittent addition of 0.1 M sodium hydroxide to facilitate a controlled reduction reaction, and wherein the vessel is sealed with a reflux condenser and continuously purged with nitrogen gas at a rate of 200 mL/min to maintain an inert atmosphere and suppress oxidative back-reactions; and wherein the heating step of the hydrazine-treated graphene oxide mixture is carried out in an oil bath equipped with a PID controller to maintain the temperature precisely between 91° C. and 94° C. for a duration of 3 hours, and wherein the reaction progress is monitored every 30 minutes by extracting 2 mL aliquots and analyzing UV-Vis absorbance at 230 nm and 265 nm to verify the progression of reduction by the decrease of oxygen-containing functional groups.

In this embodiment, the process for reducing graphene oxide (GO) to reduced graphene oxide (rGO) is executed under meticulously controlled conditions to ensure both high reduction efficiency and reproducibility, thereby fulfilling the enablement requirement and demonstrating technical efficacy. The dropwise addition of a 30% w/v hydrazine hydrate solution to the aqueous dispersion of GO is a critical parameter that allows for the gradual introduction of the reducing agent, thereby avoiding localized overheating or the formation of agglomerates that could impede reduction uniformity. The magnetic stirring at a constant 1200 rpm ensures continuous dispersion of hydrazine throughout the GO matrix, promoting consistent exposure of oxygen-functional groups on the graphene sheets to the reducing environment.

Maintaining the pH between 9.5 and 10.5 through intermittent addition of 0.1 M NaOH creates an optimal alkaline environment that accelerates the nucleophilic reduction mechanism of hydrazine while suppressing competing side reactions. This pH range is empirically determined to favor conversion of epoxide and hydroxyl groups while minimizing structural degradation of the graphene basal plane. To further maintain chemical integrity, the reaction vessel is fitted with a reflux condenser, which prevents solvent evaporation and stabilizes the reaction volume. Continuous purging with nitrogen gas at 200 mL/min establishes an inert atmosphere, which is essential for preventing re-oxidation of partially reduced GO intermediates by ambient oxygen. This gas flow rate was optimized through experimentation to effectively displace dissolved oxygen without disturbing the liquid surface.

The heating of the hydrazine-GO reaction mixture is carried out in an oil bath fitted with a PID (proportional-integral-derivative) controller that tightly regulates the temperature between 91° C. and 94° C. This temperature range is critical: temperatures below 90° C. result in incomplete reduction, while temperatures above 95° C. may cause bubble formation and possible exfoliation or fragmentation of sheets. The duration of 3 hours provides sufficient time for complete reduction, and was determined from reaction kinetics studies showing plateauing of absorbance change beyond this period. Reaction progress is quantitatively monitored using UV-Vis spectroscopy by extracting 2 mL aliquots every 30 minutes and analyzing absorbance at 230 nm and 265 nm. The absorbance peak at ˜230 nm, attributed to π-π* transitions of C═C bonds in GO, typically shifts and diminishes as conjugation is restored during reduction, while the shoulder at 265 nm associated—with n-π* transitions of C═O groups—gradually disappears, indicating loss of oxygen functionalities. For example, a typical dataset showed that after 90 minutes, the absorbance at 230 nm decreased by over 40%, and the 265 nm peak nearly vanished after 150 minutes, confirming substantial reduction.

The synergistic effect in this process arises from the concurrent optimization of multiple interdependent variables-pH control, inert atmosphere, controlled heating, and in-situ monitoring all of which contribute to the formation of high-quality rGO with fewer defects and enhanced electrical and mechanical properties. The end product, confirmed by FTIR, Raman spectroscopy, and XPS analysis, shows over 85% removal of oxygenated groups with a C/O ratio improvement from ˜2.1 to ˜6.8. The reduced graphene oxide thus produced is well-suited for integration into composite coatings due to its improved conductivity, hydrophobicity, and interfacial compatibility. This embodiment establishes a technically robust and reproducible foundation for downstream fabrication of advanced marine antifouling coatings and supports the claims with detailed procedural enablement and measurable performance metrics.

In an embodiment, after the reduction step and subsequent washing, the filtered reduced graphene oxide slurry is immersed in deionized water pre-heated to 50° C., and the dispersion is allowed to equilibrate for 30 minutes prior to sonication to aid hydration and dispersion of reduced graphene oxide sheets, and wherein the subsequent sonication is performed in a probe sonicator operating at 20 kHz with a 40% duty cycle for a continuous 6-hour period while ensuring the temperature does not exceed 30° C. by using an ice-cooled jacketed beaker; and wherein the polymer solution is prepared by dissolving 40-50% by weight of Bisphenol A-type epoxy resin in acetone, wherein the acetone is preheated to 55° C. using a reflux setup with nitrogen blanketing to avoid moisture ingress, and wherein the epoxy resin is added incrementally over 15 minutes while maintaining stirring at 1500 rpm, followed by continuous stirring for an additional 2 hours until a visually homogeneous, translucent resin solution is obtained with no particulate residues.

In this embodiment, the focus is on ensuring the optimal hydration, exfoliation, and dispersion of reduced graphene oxide (rGO) sheets, along with the preparation of a stable and homogeneous polymer matrix into which the nanofillers will be integrated. After the completion of the reduction and washing steps, the filtered rGO slurry—still rich in residual functional groups and possessing a partially re-stacked structure—is immersed in deionized water pre-heated to 50° C. The elevated temperature facilitates hydration of the rGO sheets and loosens any loosely bound aggregates by disrupting weak van der Waals interactions. This equilibration step for 30 minutes allows thermal diffusion of water into interlayer spaces, swelling the sheets and enabling better penetration during subsequent sonication.

The hydrated rGO dispersion is then subjected to probe sonication using a sonicator operating at 20 kHz and a 40% duty cycle for 6 continuous hours. The long duration of sonication ensures full exfoliation of rGO aggregates into mono- or few-layer sheets with minimal lateral stacking. However, to mitigate the heat generated by high-energy sonication—which could otherwise oxidize or fragment the rGO—the reaction is carried out in an ice-cooled, jacketed beaker, keeping the temperature consistently below 30° C. This controlled thermal environment maintains the structural integrity of rGO, preserving the sp²-hybridized carbon network while achieving superior dispersion. TEM and AFM characterization of the resulting suspension show sheet thicknesses of ˜1 nm and lateral dimensions between 0.5-2 μm, indicative of successful exfoliation.

Simultaneously, preparation of the polymer solution is carried out with equal precision. Bisphenol A-type epoxy resin is chosen due to its robust mechanical strength, thermal stability, and chemical resistance—properties desirable for marine coatings. The resin is introduced into acetone at a loading of 40-50% by weight to ensure an adequate concentration for eventual film formation while still maintaining sufficient fluidity for uniform mixing. The acetone is preheated to 55° C. using a reflux setup to expedite dissolution and reduce viscosity. Nitrogen blanketing during this step is critical to prevent moisture ingress, which could otherwise lead to unwanted hydrolysis or premature cross-linking of the epoxy.

The epoxy resin is added incrementally over a period of 15 minutes while the system is stirred continuously at 1500 rpm. This gradual addition avoids clumping or localized concentration gradients that might otherwise cause inhomogeneous polymer dispersion. After full resin addition, stirring is continued for an additional 2 hours to allow full solubilization and homogenization. The final mixture is visually inspected and appears as a translucent, slightly viscous liquid with no sediment or haze under optical microscopy, indicating complete dissolution and the absence of particulate impurities. Rheological studies on the resulting solution reveal Newtonian behavior with viscosity in the range of 900-1200 cP at 25° C., suitable for both casting and spray-coating methods.

The technical efficacy of this embodiment lies in the dual optimization: the well-dispersed rGO sheets ensure maximized surface area for mechanical reinforcement and barrier effects, while the homogeneous epoxy-acetone solution ensures uniform embedding of these sheets without phase separation or aggregation during mixing and curing. Moreover, this precise control over nanofiller dispersion and matrix preparation directly contributes to the synergistic performance of the final coating system, as later evidenced by improved water barrier properties, higher tensile strength, and enhanced antifouling characteristics. For example, samples prepared using this method showed up to 40% improvement in tensile modulus and over 90% reduction in water uptake compared to control epoxy coatings without nanofillers. This confirms both the reproducibility and effectiveness of the described embodiment and its critical role in realizing the technical advantages claimed in the invention.

In an embodiment, before mixing with the polymer solution, the reduced graphene oxide dispersion is subjected to pH adjustment using 0.05 M NaOH solution to bring the pH between 6.8 and 7.2 to ensure compatibility with the epoxy resin, and wherein the dispersion is then homogenized at 8000 rpm for 10 minutes using a rotor-stator mixer to eliminate any residual flake clusters and ensure interfacial compatibility between the graphene phase and the organic matrix; and wherein the zinc oxide nanoparticles are first suspended in ethanol and sonicated at 40 kHz for 1 hour, then modified using 0.3% v/v 3-glycidyloxypropyltrimethoxysilane (GPTMS) at 60° C. for 2 hours under reflux to introduce epoxy-functional surface groups, and the modified ZnO particles are then vacuum-dried at 70° C. for 6 hours to yield functionalized nanoparticles that form covalent bonds with the epoxy matrix, improving nanoparticle dispersion and long-term antifouling performance.

In this embodiment, critical preparatory steps are undertaken to ensure the chemical compatibility and stable dispersion of nanomaterials-specifically reduced graphene oxide (rGO) and zinc oxide (ZnO) nanoparticles within the epoxy matrix, thereby enabling the formation of a structurally integrated, high-performance antifouling coating. These steps directly support the enablement requirement by detailing conditions and parameters that yield reproducible, technically effective results.

The first stage involves adjusting the pH of the rGO dispersion prior to mixing with the epoxy polymer solution. The as-prepared rGO typically possesses a mildly acidic pH (around 5.5-6.0) due to residual oxygen-containing functional groups such as carboxyl and phenolic moieties. If left unneutralized, this acidity can interfere with the polymerization of epoxy resins, leading to premature curing, phase separation, or poor interface adhesion. Therefore, 0.05 M NaOH is added dropwise under stirring until the pH reaches a physiologically compatible range of 6.8 to 7.2. This controlled neutralization not only stabilizes the dispersion but also ensures that the chemical environment aligns with the epoxy resin's curing kinetics. Empirical trials confirm that coatings produced with adjusted pH dispersions exhibit a 20-25% improvement in tensile strength and adhesion compared to those using unadjusted rGO.

Following pH normalization, the dispersion is homogenized using a rotor-stator mixer operating at 8000 rpm for 10 minutes. The high shear forces generated in this equipment serve to break apart any residual rGO aggregates and flake clusters, which can otherwise result in weak zones and poor stress transfer in the final coating. Post-homogenization analysis using dynamic light scattering (DLS) and zeta potential measurements typically shows a monodisperse distribution of rGO flakes with hydrodynamic diameters centered around 300-400 nm and zeta potential values exceeding ±30 mV, indicating colloidal stability suitable for long-term storage and formulation.

In parallel, the zinc oxide nanoparticles undergo a dual-phase treatment process. First, the ZnO nanoparticles are dispersed in absolute ethanol and sonicated at 40 kHz for 1 hour to ensure their deagglomeration and surface activation. Ethanol acts as a polar medium that not only aids dispersion but also supports the subsequent silanization reaction. The sonicated ZnO dispersion is then treated with 0.3% v/v 3-glycidyloxypropyltrimethoxysilane (GPTMS) under reflux at 60° C. for 2 hours. GPTMS molecules undergo hydrolysis and condensation, forming covalent Si—O—Zn bonds with the nanoparticle surfaces and exposing reactive epoxide terminal groups on the outer surface.

This functionalization process chemically links ZnO to the epoxy matrix during curing, forming robust covalent interfaces that inhibit nanoparticle migration, reduce agglomeration during thermal curing, and enhance mechanical integrity. FTIR spectroscopy of the functionalized ZnO reveals characteristic absorption peaks near 910 cm⁻¹ and 1250 cm⁻¹ corresponding to epoxide and siloxane groups, respectively. After reflux, the particles are vacuum-dried at 70° C. for 6 hours to remove residual ethanol and drive off unreacted silane, yielding dry, surface-functionalized ZnO nanoparticles with enhanced stability and storage longevity.

When integrated into the epoxy matrix, these epoxy-reactive ZnO nanoparticles form strong interfacial linkages with the resin during the thermal curing process, contributing both antimicrobial and structural reinforcement effects. SEM and EDS elemental mapping of cured films show uniform ZnO distribution and no significant clustering, even after 6 months of shelf storage. Further, antifouling tests conducted in simulated marine biofouling conditions show over 85% reduction in microbial adhesion compared to unmodified ZnO controls, indicating superior long-term fouling resistance. The synergistic advantage of this embodiment lies in the interplay between properly dispersed, pH-balanced rGO providing barrier and mechanical reinforcement, and chemically grafted ZnO offering antimicrobial properties and stable interface anchoring. These engineered interfaces and controlled processing steps result in reproducible, high-performing coatings that meet industrial durability, hydrophobicity, and antifouling benchmarks, thus rendering the process fully enabled and technically validated.

In an embodiment, the carbon nanotubes are first refluxed in a 3:1 volume ratio mixture of concentrated nitric acid and sulfuric acid at 90° C. for 4 hours, followed by repeated washing with deionized water until the pH of the supernatant stabilizes at 7.0, and vacuum drying at 60° C. for 12 hours, to introduce carboxyl and hydroxyl functional groups on the nanotube surfaces, which enhance chemical compatibility with the epoxy resin and promote stronger interfacial adhesion in the composite; and wherein the formation of the composite material comprises sequentially adding the reduced graphene oxide solution, functionalized zinc oxide nanoparticles, and acid-treated carbon nanotubes into the epoxy-acetone polymer solution, with each component addition followed by 30 minutes of mechanical stirring at 1000 rpm and 1 hour of bath sonication at 40 kHz, and wherein the final mixture is subjected to probe sonication at 20 kHz for 1 hour to ensure nanoscale uniformity before coating.

In this embodiment, the focus is on achieving superior interfacial integration of carbon nanotubes (CNTs) within a nanocomposite epoxy matrix, in synergy with reduced graphene oxide (rGO) and functionalized zinc oxide (ZnO) nanoparticles, to produce a homogeneous, high-performance marine coating. The process begins with functionalization of the CNTs to improve their compatibility with the epoxy polymer system. Raw multi-walled CNTs are refluxed in a strongly oxidizing acid mixture composed of concentrated nitric acid and sulfuric acid in a 3:1 volume ratio at 90° C. for 4 hours. This acid treatment introduces oxygen-containing surface functional groups—primarily carboxyl (—COOH) and hydroxyl (—OH) moieties—on the CNT walls and open ends through oxidative cleavage and surface etching. These groups increase the polarity and dispersibility of CNTs in polar solvents and resin systems and create active sites for covalent and hydrogen bonding with the epoxy matrix, leading to stronger interfacial adhesion.

Following acid treatment, the CNTs are thoroughly washed with deionized water multiple times until the wash supernatant reaches a neutral pH of 7.0, ensuring complete removal of residual acids that could catalyze unwanted side reactions or interfere with resin curing. The washed CNTs are then vacuum-dried at 60° C. for 12 hours to remove moisture without degrading the newly introduced functional groups. Characterization by FTIR shows broad —OH stretching vibrations and C═O peaks indicative of —COOH groups, while zeta potential measurements reveal improved dispersion stability in polar solvents (±35 mV or greater). TEM imaging confirms reduced bundling compared to untreated CNTs, and TGA analysis reveals the presence of surface oxygenated species accounting for ˜10 wt % mass loss attributable to functional groups.

To form the nanocomposite coating, a stepwise integration method is employed. The rGO dispersion, previously prepared and sonicated, is first introduced into the epoxy-acetone polymer solution under controlled conditions, followed by functionalized ZnO nanoparticles, and finally, the acid-treated CNTs. This order is critical to avoid competitive interactions between fillers and to ensure hierarchical organization within the matrix. After each filler addition, the mixture is mechanically stirred at 1000 rpm for 30 minutes to promote initial distribution, followed by bath sonication at 40 kHz for 1 hour to assist de-agglomeration and uniform dispersion at the microscale.

Upon addition of the final component—acid-treated CNTs—the complete nanocomposite mixture is subjected to high-energy probe sonication at 20 kHz for 1 hour. This step is crucial for achieving nanoscale uniformity, minimizing interfacial voids, and enhancing the connectivity of filler networks within the epoxy matrix. The synergistic effect arises from the complementary roles of each nanofiller: rGO offers a barrier to moisture and gas permeation, ZnO provides antimicrobial functionality, and CNTs impart tensile reinforcement and electrical conductivity. The acid functionalization enables strong interfacial bonding, which not only improves load transfer and reduces filler pull-out under stress but also prevents phase segregation during curing.

Experimental results from scanning electron microscopy (SEM) and transmission electron microscopy (TEM) confirm uniform dispersion of all three nanofillers, with no visible micron-scale agglomerates. Mechanical testing of cured films shows a ˜35% increase in tensile strength and a 45% increase in modulus compared to neat epoxy. Additionally, electrochemical impedance spectroscopy (EIS) reveals enhanced barrier properties, with coating resistance values an order of magnitude higher than control samples, indicating improved corrosion resistance. These results collectively validate the technical efficacy of the embodiment and establish the enablement of a reproducible, high-performance coating system achieved through carefully orchestrated chemical functionalization, dispersion, and nanocomposite formulation.

In an embodiment, during the sonication of the composite mixture, the temperature is monitored every 10 minutes using an immersed thermocouple and maintained below 35° C. by placing the container in an ice-water bath, and wherein the mixture is allowed to rest for 1 hour post-sonication to degas entrapped air bubbles, enabling the formation of a dense, pinhole-free coating film upon application to the substrate; and wherein the coating is applied by spray coating using a two-stage airbrush system with a 0.5 mm nozzle diameter, operated at 2 bar air pressure, and wherein three successive coating layers are applied with 10-minute intervals between each layer, followed by drying under ambient conditions for 12 hours and curing at 30° C. and 40% relative humidity for 48 hours to achieve an adherent, crack-free, and flexible coating.

In this embodiment, the focus is on ensuring the thermal integrity, structural homogeneity, and defect-free film formation of the composite coating during the final stages of its preparation and application. These steps are pivotal for translating the nanoscale dispersion of active ingredients into a macroscopically uniform, durable, and high-performance antifouling layer. The process begins with controlled sonication of the complete composite mixture-which includes reduced graphene oxide (rGO), functionalized ZnO nanoparticles, and acid-treated carbon nanotubes-dispersed within the epoxy-acetone solution. To prevent degradation of polymer chains or destabilization of nanofiller dispersion due to localized heating, the sonication is conducted under strict thermal monitoring. An immersed thermocouple continuously measures the temperature of the reaction mixture, with readings taken every 10 minutes. The container is placed in an ice-water bath throughout the 1-hour probe sonication (20 kHz) session, effectively maintaining the temperature below 35° C.

Maintaining this low temperature is essential, as excessive heat during sonication could trigger premature crosslinking reactions in the epoxy resin or promote nanoparticle agglomeration, both of which would compromise the coating's uniformity and performance. Moreover, by preventing thermal damage to the chemically functionalized fillers, the interfacial bonding between the filler and the epoxy matrix is preserved, which is vital for mechanical integrity and long-term durability. Following sonication, the mixture is allowed to rest undisturbed for 1 hour. This degassing period allows entrapped air bubbles, which may have formed due to high-energy agitation, to escape. Eliminating these voids is crucial for producing a dense, pinhole-free coating film that ensures effective barrier properties and resistance to water, oxygen, and microbial penetration.

The degassed, homogenous mixture is then applied onto prepared substrates using a two-stage airbrush spray coating system fitted with a 0.5 mm nozzle and operated at a constant air pressure of 2 bar. The nozzle diameter and air pressure are optimized for balancing atomization efficiency and film thickness control. This precision-controlled spray mechanism ensures fine, even distribution of the nanocomposite coating across the substrate surface. Three successive layers are applied, each spaced 10 minutes apart to allow partial drying and surface tackiness to develop, which improves interlayer adhesion and prevents solvent entrapment.

Post-application, the coated substrate is left to dry under ambient conditions for 12 hours to allow gradual solvent evaporation, followed by a low-temperature curing protocol at 30° C. and 40% relative humidity for 48 hours. This curing environment is carefully chosen to avoid thermal stress or rapid solvent off-gassing, which could cause cracks or delamination. Instead, it facilitates slow and uniform cross-linking of the epoxy matrix, thereby forming a flexible, crack-free, and adherent film. Optical microscopy and SEM imaging of the final coating confirm the absence of visible pores, blisters, or surface roughness inconsistencies, while tape adhesion tests (ASTM D3359) reveal a 5B rating-indicative of excellent adhesion. This embodiment illustrates a synergy between process control and material performance. The thermal regulation during sonication preserves filler integrity and dispersion, while degassing prevents voids that would compromise the film's barrier properties. The optimized multi-layer spray application yields a uniform thickness distribution (typically 50-60 μm per coat) and smooth finish, essential for minimizing surface fouling and ensuring mechanical resilience in marine environments. Coated substrates tested under accelerated aging and immersion in artificial seawater for 60 days exhibit no delamination or fouling beyond 10% surface area-demonstrating the real-world efficacy of this process and its reproducible, scalable applicability.

In an embodiment, prior to coating application, the substrate is pre-treated by sandblasting with 120-grit alumina at 60 psi, followed by ultrasonication in acetone for 15 minutes and drying under vacuum at 50° C. for 1 hour, to create a micro-roughened, clean surface that enhances mechanical interlocking and chemical adhesion between the substrate and the coating; and wherein the prepared coating composition is evaluated before application using zeta potential analysis and dynamic light scattering to confirm that the particle dispersion exhibits a zeta potential greater than ±30 mV and particle size distribution centered around 150 nm with a polydispersity index below 0.2, indicating excellent colloidal stability suitable for uniform marine coatings.

In this embodiment, surface preparation of the substrate and pre-application characterization of the nanocomposite coating formulation are strategically employed to ensure optimal coating adhesion, uniformity, and long-term performance in harsh marine environments. These preparatory steps are not only essential from a materials engineering standpoint but are also critical in fulfilling the enablement requirement, as they define reproducible parameters that directly influence the efficacy of the final antifouling coating.

The substrate—typically a metal such as marine-grade steel or aluminum—is first subjected to mechanical surface activation via sandblasting using 120-grit alumina particles at a pressure of 60 psi. This grit size and pressure combination was experimentally optimized to create a micro-roughened surface morphology without causing surface deformation or microcracks. The micro-texturing significantly increases the surface area and introduces anchor sites that enhance mechanical interlocking between the substrate and the subsequently applied epoxy-based nanocomposite. Surface profilometry measurements indicate a roughness average (Ra) in the range of 0.5-1.2 μm, which has been shown to correlate with improved coating adhesion and resistance to delamination under shear stress.

Following mechanical abrasion, the substrate is ultrasonically cleaned in acetone for 15 minutes. This step removes residual particulate matter, oil, and other organic contaminants introduced during sandblasting. Ultrasonication enhances the penetration of acetone into surface crevices, enabling efficient cleaning at the micro and nanoscale. The cleaned substrate is then dried under vacuum at 50° C. for 1 hour to remove any solvent residues and adsorbed moisture, thereby preventing the formation of interfacial voids or blisters during coating application and curing.

In parallel, the nanocomposite coating composition—consisting of reduced graphene oxide (rGO), functionalized ZnO nanoparticles, and acid-treated carbon nanotubes dispersed in an epoxy-acetone solution—is evaluated to verify its colloidal stability and suitability for spray application. Two key analytical techniques are employed: zeta potential analysis and dynamic light scattering (DLS). Zeta potential is a measure of the surface charge and electrostatic repulsion between particles in suspension. Values greater than ±30 mV are indicative of strong electrostatic stabilization, minimizing the risk of agglomeration and phase separation. The coating dispersion prepared in this embodiment consistently exhibits zeta potentials in the range of +38 to +45 mV, confirming its high colloidal stability even under shear and thermal fluctuations associated with the spray-coating process.

Dynamic light scattering further quantifies the particle size distribution of the nanocomposite system. A narrow distribution centered around 150 nm with a polydispersity index (PDI) below 0.2 is achieved, demonstrating that the fillers are uniformly dispersed at the nanoscale without the presence of large aggregates or multiple size populations. This level of dispersion quality is critical for forming defect-free films and ensuring uniform thickness, hydrophobicity, and mechanical performance across large surface areas. TEM imaging corroborates these findings, showing well-dispersed rGO sheets, isolated ZnO particles, and uniformly distributed CNTs within the polymer matrix. The synergistic impact of these pre-treatment and characterization steps is substantial. The roughened, clean substrate surface promotes strong adhesion through both chemical bonding (via residual functional groups on the nanofillers) and mechanical interlocking. Simultaneously, the stable and uniformly dispersed coating solution enables consistent deposition, curing, and film formation without sagging, cracking, or phase segregation. Coatings applied under these conditions show a pull-off adhesion strength of ≥3.5 MPa (ASTM D4541), and exhibit no delamination or blistering after 1000 hours of salt spray exposure (ASTM B117). These results provide concrete evidence of the technical efficacy and reproducibility of this embodiment and its critical role in achieving a durable and high-performance marine antifouling coating system.

In an embodiment, the coated substrate is tested for antifouling efficacy by immersion in a simulated marine environment comprising 3.5% NaCl, marine algae spores, and bacteria cultures for a period of 30 days, and wherein the coating resists biofilm formation with surface microbial coverage below 10% as observed under SEM, in comparison to greater than 70% surface fouling on an uncoated control; and wherein the final cured coating is tested for water contact angle and exhibits a contact angle greater than 125°, indicating superhydrophobic surface behavior due to the combined micro/nano surface roughness generated by ZnO and CNTs and the low surface energy of the cured epoxy matrix reinforced by rGO sheets.

In this embodiment, the antifouling performance and surface characteristics of the cured nanocomposite coating are rigorously evaluated to demonstrate its functional efficacy under simulated marine conditions. These performance validations are critical to establishing the utility and technical advantage of the invention and ensure that the claimed process yields a reproducibly effective antifouling solution.

After complete curing under controlled humidity and temperature conditions, the coated substrates are immersed in a laboratory-simulated marine environment designed to mimic coastal seawater exposure. The immersion medium consists of 3.5% sodium chloride (NaCl) to simulate salinity equivalent to natural seawater, and is enriched with a biologically relevant mix of marine algae spores and bacterial cultures known to contribute to early-stage biofouling. The test duration is set to 30 days, during which the samples are kept under constant circulation and ambient lighting to replicate conditions that promote microbial colonization and biofilm development.

Post-immersion analysis of the coated substrates is conducted using scanning electron microscopy (SEM) to visualize and quantify microbial colonization. The results consistently show that the coating resists biofilm formation, with microbial surface coverage remaining below 10%. In contrast, uncoated control substrates exposed under identical conditions exhibit dense biofouling with over 70% surface coverage by bacterial clusters and algal films. This substantial reduction in fouling confirms the active antifouling efficacy of the nanocomposite system, which is attributed to multiple, synergistic mechanisms: the antimicrobial action of ZnO nanoparticles, the low surface energy imparted by the epoxy matrix and PDMS oligomer (if used), and the nanoscale texture provided by well-dispersed CNTs and rGO sheets.

Furthermore, the surface wettability of the cured coating is characterized by static water contact angle measurement. The coating consistently exhibits a contact angle greater than 125°, qualifying it as superhydrophobic. This high contact angle indicates that water and marine bio-organisms are less likely to adhere and spread across the surface, a critical characteristic for fouling-release coatings. The superhydrophobic behavior arises from the combined effect of nanoscale surface roughness—imparted by the hierarchical distribution of ZnO and CNTs—and the inherently low surface energy of the cured epoxy matrix, which is further enhanced by the embedded rGO nanosheets that align parallel to the surface during curing.

Atomic force microscopy (AFM) scans of the coating reveal a surface roughness (Ra) in the range of 150-250 nm, which is ideal for maintaining a Cassie-Baxter wetting regime that promotes water repellency and reduces microorganism adhesion. The antifouling performance is further validated through fluorescence-based live/dead staining assays, which confirm minimal viable bacterial colonization compared to controls. These performance traits are not incidental but result from the synergistic interplay between chemically functionalized nanofillers, precisely controlled dispersion, tailored surface morphology, and optimized curing parameters. Together, these features not only confirm the coating's technical efficacy under prolonged marine exposure but also substantiate its reproducibility, scalability, and industrial relevance for long-term marine protection applications.

In an embodiment, the adhesion strength of the coating on the substrate is evaluated using pull-off testing and exhibits an average adhesion strength of 3.5 MPa, which is attributed to the dual contribution of functionalized nanoparticle-epoxy interactions and the intercalated rGO structure that forms a continuous load transfer network; and wherein the mechanical integrity of the coating is evaluated using nanoindentation tests and shows a hardness of 0.25 GPa and reduced modulus of 4.8 GPa, and wherein the coating withstands 1000 hours of salt spray testing (ASTM B117) without visible blistering, peeling, or corrosion at the interface, demonstrating its long-term durability and protective antifouling performance under simulated marine conditions.

In this embodiment, the structural robustness and long-term durability of the nanocomposite coating are demonstrated through a series of standardized mechanical and environmental tests, validating both the efficacy and industrial applicability of the claimed process. The coating, prepared via the dispersion and integration of reduced graphene oxide (rGO), functionalized zinc oxide (ZnO) nanoparticles, and acid-treated carbon nanotubes (CNTs) into an epoxy matrix, is applied onto a properly pretreated substrate and allowed to cure under controlled conditions. Post-curing, the adhesion strength of the coating to the substrate is quantitatively evaluated using pull-off adhesion testing as per ASTM D4541. The coating exhibits an average adhesion strength of 3.5 MPa, indicating strong bonding between the coating and the substrate.

This superior adhesion is primarily attributed to two synergistic factors. First, the use of silane-functionalized ZnO nanoparticles introduces epoxy-reactive sites on the filler surface, which form covalent bonds with the surrounding matrix during curing. This chemical bonding at the interface enhances filler retention, stress transfer, and matrix continuity. Second, the presence of rGO sheets, exfoliated and well-dispersed throughout the matrix, forms an intercalated network that acts as a continuous load transfer pathway. This network mitigates crack propagation by deflecting stress and distributing mechanical loads evenly across the coating layer. Cross-sectional SEM and EDX mapping confirm uniform filler dispersion and absence of delamination or interfacial voids—evidence of strong interfacial cohesion and structural integration.

To further evaluate the mechanical integrity of the cured film, nanoindentation tests are conducted using a Berkovich indenter. The measurements yield a hardness value of 0.25 GPa and a reduced modulus of 4.8 GPa. These values represent a significant enhancement over unmodified epoxy coatings, which typically show hardness in the range of 0.15-0.18 GPa and reduced modulus around 3.0 GPa. The increase in hardness and stiffness is due to the load-bearing contribution of the nanofillers, especially the rGO sheets and CNTs, which act as reinforcing agents within the polymeric matrix and prevent localized deformation under stress. The mechanical data confirm that the coating not only adheres well but also maintains structural integrity under mechanical loading—an essential requirement for marine and industrial applications.

The environmental durability of the coating is assessed through salt spray testing (ASTM B117), a widely accepted accelerated corrosion test simulating long-term exposure to saline atmospheres. The coated samples are exposed to a continuous salt fog environment for 1000 hours. Post-exposure inspection reveals no signs of blistering, peeling, rusting, or interface corrosion on the coated surfaces. In contrast, control samples without nanofiller reinforcement show visible underfilm corrosion, blistering, and delamination after just 300-400 hours. The rGO component plays a particularly crucial role in this durability by introducing tortuous diffusion paths that hinder the ingress of corrosive species such as chloride ions and moisture. In combination with the impermeability of CNTs and the biocidal activity of ZnO nanoparticles, the coating demonstrates multifunctional protection against both biofouling and chemical degradation. The synergistic interplay between chemically functionalized nanoparticles and rGO-reinforced matrix structure yields a tough, adherent, and durable coating system capable of withstanding prolonged exposure to aggressive marine environments. These results not only reinforce the technical advantages of the claimed invention but also enable industrial translation and real-world application in sectors such as marine transportation, offshore infrastructure, and subsea equipment protection.

In an embodiment, prior to mixing with the polymer solution, the reduced graphene oxide dispersion is subjected to freeze-drying at −50° C. under vacuum for 24 hours to obtain rGO powder, and wherein the dried rGO is subsequently redispersed in acetone at 5 mg/mL concentration using probe sonication at 20 kHz for 30 minutes, enabling solvent-phase dispersion of rGO directly into the epoxy-acetone solution, which improves interfacial compatibility and reduces interfacial voids in the cured coating, and wherein the relative proportions of reduced graphene oxide, zinc oxide nanoparticles, and carbon nanotubes are adjusted such that the total nanofiller content remains below 40% by weight of the polymer matrix, and wherein the selected ratio of 2:1:1 between rGO, ZnO, and CNTs is maintained to maximize synergistic reinforcement, wherein rGO provides barrier properties, ZnO offers antimicrobial activity, and CNTs enhance tensile strength and conductivity within the cured composite.

In this embodiment, the process focuses on optimizing the dispersion quality and synergistic reinforcement of nanofillers within the epoxy matrix by employing a freeze-drying and solvent-phase redispersion approach for reduced graphene oxide (rGO), and by maintaining a strategically balanced ratio of nanofillers. These steps are crucial not only for improving interfacial compatibility and filler distribution but also for ensuring the final coating exhibits enhanced mechanical, antifouling, and barrier properties, thereby meeting the enablement requirement through reproducible and technically beneficial process parameters.

Initially, the rGO dispersion obtained from the reduction process is subjected to freeze-drying at −50° C. under vacuum for 24 hours. Freeze-drying is a low-temperature dehydration technique that removes water via sublimation while preserving the planar sheet morphology and structural integrity of rGO. Unlike high-temperature drying methods, freeze-drying prevents thermal degradation and minimizes aggregation due to capillary collapse, yielding a lightweight rGO aerogel or powder with high surface area and re-dispersibility. The resulting rGO powder is characterized by Raman spectroscopy, showing a prominent D-to-G band intensity ratio (I_D/I_G≈1.0), indicating partial reduction with abundant defect sites suitable for interfacial anchoring in polymer matrices.

The dried rGO is then redispersed in acetone at a concentration of 5 mg/mL using probe sonication at 20 kHz for 30 minutes. Acetone serves as an ideal solvent due to its polarity and compatibility with the epoxy resin. The sonication facilitates exfoliation and homogeneous dispersion of rGO sheets, which is critical for achieving solvent-phase blending with the epoxy-acetone solution. This approach—dispersing rGO in the same solvent as the polymer—not only improves interfacial compatibility by reducing solvent-resin polarity mismatch but also minimizes phase separation and interfacial voids during the curing stage. SEM and TEM analyses confirm a well-exfoliated and uniformly distributed rGO phase in the cured matrix, with no micron-scale agglomerates or dry zones, which are commonly observed in water-dried rGO.

To further ensure process reproducibility and optimized performance, the total nanofiller loading is maintained below 40% by weight of the polymer matrix. This threshold is established based on rheological and mechanical testing that shows higher filler loadings lead to increased viscosity, reduced sprayability, and the formation of brittle films due to poor wetting and incomplete cross-linking. The nanofillers—rGO, ZnO, and CNTs—are incorporated in a controlled weight ratio of 2:1:1. This ratio has been experimentally determined to provide a balanced set of properties by leveraging the unique functionalities of each component. Specifically, rGO serves as a planar barrier phase, reducing oxygen and water vapor transmission rates through a tortuous path mechanism. ZnO nanoparticles contribute antimicrobial properties through reactive oxygen species (ROS) generation and direct bacterial membrane disruption. CNTs impart tensile reinforcement, improving the film's toughness and resistance to mechanical abrasion, while also enhancing electrical conductivity and crack-bridging capability.

The synergistic behavior of this 2:1:1 ratio is substantiated through comparative tests. Coatings prepared with this ratio demonstrate a ˜60% improvement in tensile strength and a ˜70% increase in Young's modulus compared to neat epoxy coatings. Water vapor transmission rate (WVTR) is reduced by over 80%, and contact angle measurements consistently exceed 125°, indicating superhydrophobicity. Microbial fouling studies conducted over 30 days in simulated marine environments show <10% surface coverage, outperforming coatings with imbalanced or single-filler compositions. Electrochemical impedance spectroscopy (EIS) reveals high coating resistance and low charge transfer values, consistent with superior barrier integrity and long-term corrosion resistance. This embodiment ensures that the structural, chemical, and functional integration of rGO, ZnO, and CNTs is optimized for maximum synergistic effect. The freeze-drying and redispersion strategy for rGO not only preserves its morphology but also enables its seamless integration into the epoxy system, while the strict control of filler ratios and total loading ensures process reproducibility, coating uniformity, and multi-functional performance across mechanical, antifouling, and barrier domains. These elements collectively support the enablement and inventive step of the claimed process.

In an embodiment, the epoxy-based coating mixture exhibits a shear-thinning behavior characterized by a decrease in viscosity from 1200 cP at 10 s⁻¹ to 400 cP at 100 s⁻¹ as measured by a rotational rheometer at 25° C., and wherein such rheological behavior allows ease of application by spraying while maintaining sag resistance and thickness uniformity on vertical marine surfaces during curing; and wherein the reduced graphene oxide, ZnO, and CNTs are co-dispersed in a pre-mix stage with 2% by weight non-ionic surfactant selected from the group consisting of Triton X-100 or Pluronic F-127, and wherein the pre-mix is subjected to bath sonication for 2 hours before being introduced into the epoxy solution, thereby enhancing inter-particle spacing and preventing filler aggregation during mixing and curing.

In this embodiment, the focus is placed on the rheological optimization and pre-dispersion strategy of the nanofillers—reduced graphene oxide (rGO), zinc oxide (ZnO) nanoparticles, and carbon nanotubes (CNTs)—within the epoxy matrix to achieve excellent sprayability, coating uniformity, and long-term stability. These process-specific details directly contribute to the technical efficacy of the final antifouling coating and serve to fulfill the enablement requirement by ensuring repeatable performance and applicability under industrial conditions, particularly in the marine sector where vertical or curved surface coating is routine.

The epoxy-based nanocomposite coating mixture is designed to exhibit shear-thinning behavior, a non-Newtonian flow characteristic wherein viscosity decreases with increasing shear rate. Rheological measurements using a rotational rheometer at 25° C. reveal a viscosity of approximately 1200 centipoise (cP) at a low shear rate of 10 s⁻¹, which decreases steadily to 400 cP at a high shear rate of 100 s⁻¹. This rheological profile is ideal for spray application: under the high shear conditions imparted by spray nozzles, the reduced viscosity allows the coating to atomize into fine droplets and flow smoothly across the substrate. Once deposited, the lower shear environment causes the viscosity to increase again, allowing the coating to resist sagging and maintain film thickness uniformity-particularly important for vertical or overhead surfaces.

This shear-thinning behavior is engineered through a careful balance of polymer-solvent interactions and nanofiller dispersion. To ensure homogeneous filler integration and prevent aggregation, the rGO, ZnO, and CNTs are first subjected to a co-dispersion or pre-mix step. In this step, the nanofillers are mixed in a common solvent-typically acetone or ethanol-along with 2% by weight of a non-ionic surfactant. The surfactant is selected from the group consisting of Triton X-100 or Pluronic F-127, both of which are known to stabilize nanomaterials by providing steric hindrance and hydrophilic-lipophilic balance. Triton X-100, a polyethylene glycol-based surfactant, adsorbs onto the surfaces of rGO and CNTs, creating a repulsive layer that prevents Van der Waals-driven re-agglomeration. Pluronic F-127, a triblock copolymer, further enhances dispersion through micelle formation and hydrophobic-hydrophilic domain segregation.

The pre-mix is then subjected to bath sonication for 2 hours. This step promotes deagglomeration, exfoliation (in the case of rGO), and uniform surfactant adsorption onto the particle surfaces. It also increases inter-particle spacing and disperses fillers in a thermodynamically stable manner, minimizing sedimentation or phase separation upon mixing with the epoxy solution. Dynamic light scattering (DLS) and zeta potential analysis confirm that the sonicated premix achieves a narrow particle size distribution (˜150-200 nm) with a polydispersity index below 0.2 and zeta potential values exceeding ±30 mV, indicating colloidal stability.

Once the stabilized pre-mix is introduced into the epoxy-acetone solution, it blends seamlessly without observable flocculation or phase mismatch. This leads to a uniform coating solution that can be spray-applied using standard equipment while maintaining optimal thickness (typically 50-60 μm per coat), mechanical integrity, and surface smoothness. Visual inspection under optical microscopy shows no streaks or sags, even when applied to vertical marine-grade steel panels. Moreover, coatings formed under these conditions exhibit consistent superhydrophobic behavior (contact angle >125°), high adhesion strength (˜3.5 MPa), and antifouling resistance (<10% microbial coverage after 30-day marine immersion). Thus, this embodiment underscores the significance of tailoring the rheology and nanofiller dispersion chemistry to produce a process-friendly, functionally robust coating formulation. The co-dispersion of rGO, ZnO, and CNTs in the presence of a carefully selected surfactant system, coupled with bath sonication and precise rheological control, ensures not only easy application but also high-performance coating characteristics—demonstrating a strong synergy between materials science and process engineering in the realization of the invention.

In an embodiment, the prepared coating is applied to underwater steel structures using brushing, and wherein after each coat, the surface is flash-dried using IR lamps at 40° C. for 10 minutes prior to the next coat application, and wherein a total of three coats are applied with an average thickness of 50-60 μm per coat as measured by eddy current thickness gauge, resulting in a multi-layer barrier structure that improves long-term antifouling resistance.

In this embodiment, the application technique and layer-by-layer deposition strategy are designed to ensure reliable coating performance on underwater steel structures, particularly in marine or offshore environments where corrosion and biofouling are persistent concerns. The use of manual brushing as the application method highlights the coating's versatility and adaptability to non-uniform, complex geometries, such as ship hulls, subsea pipelines, or dock pilings, where spray-based application may be impractical due to constraints like access, environmental conditions, or safety regulations.

The nanocomposite epoxy-based coating—comprising reduced graphene oxide (rGO), functionalized zinc oxide (ZnO) nanoparticles, and acid-treated carbon nanotubes (CNTs)—is prepared as a stable dispersion in an epoxy-acetone matrix with rheological properties suitable for both brushing and self-leveling. The viscosity and surface wetting properties are tuned to allow smooth spreading without brush marks, enabling even deposition over rough, irregular steel surfaces. The brushing process ensures that the coating reaches corners, weld seams, bolt interfaces, and other structural discontinuities where fouling or corrosion typically initiates.

After each coat is applied, it is flash-dried using infrared (IR) lamps at a controlled temperature of 40° C. for 10 minutes. This intermediate drying step plays a critical role in solvent evaporation and initial resin gelation, preventing solvent entrapment, bubble formation, or layer delamination in subsequent coatings. IR drying at 40° C. is selected because it is above the acetone evaporation point yet below the thermal degradation temperature of the resin and nanofillers, ensuring rapid but controlled layer stabilization. Infrared heating also promotes surface cross-linking, which increases the interfacial adhesion between layers without requiring full thermal curing at each stage. This staged approach results in strong interlayer bonding and suppresses crack formation or mechanical decoupling during the curing process.

A total of three coats are sequentially applied using this method, each with an average dry film thickness of 50-60 μm. The total coating thickness thus ranges from 150 to 180 μm, which has been experimentally determined to balance flexibility, barrier performance, and mechanical strength. Thickness is verified using a calibrated eddy current thickness gauge, a non-destructive measurement technique particularly suitable for metallic substrates with non-conductive coatings. This precise layer control ensures uniformity across large surfaces and mitigates localized thinning, which could compromise performance.

The resulting multi-layer barrier structure creates a tortuous path for water molecules, salts, and oxygen, significantly impeding diffusion and corrosion propagation. The rGO sheets form a stacked lamellar network that enhances impermeability, while the embedded CNTs bridge across layers and cracks to improve tensile integrity and suppress delamination. ZnO nanoparticles distributed across all three layers provide sustained antimicrobial activity and biofilm resistance.

The technical efficacy of this layered architecture is demonstrated in salt spray and biofouling tests. Samples coated with three brushed layers show no visible blistering, peeling, or rust spots after 1000 hours of ASTM B117 salt spray exposure. In fouling resistance assays, these samples show <15% surface microbial coverage compared to >80% in single-layer or uncoated controls. Moreover, the multilayered structure exhibits improved self-healing under cyclic thermal stress due to the presence of overlapping graphene and CNT networks that redistribute strain and preserve coating continuity. This embodiment ensures process scalability and field applicability while maintaining the integrity and functionality of the nanocomposite coating. The sequential brushing and IR flash-drying protocol, coupled with consistent thickness control, lead to a multilayer coating that exhibits superior antifouling resistance, corrosion protection, and mechanical durability—satisfying both the enablement and performance requirements for real-world underwater applications.

In an embodiment, after final curing, the coated surface exhibits an oxygen transmission rate (OTR) less than 1.5 cm³/m²/day at 25° C. and 1 atm, as measured by a gas permeability tester, wherein the layered tortuosity introduced by the aligned reduced graphene oxide sheets embedded in the epoxy network impedes gas diffusion pathways and enhances corrosion protection under marine immersion conditions; and wherein the coating exhibits a surface roughness (Ra) of 150-300 nm as determined by atomic force microscopy (AFM), and wherein this surface texture is governed by the size and distribution of embedded ZnO and CNTs, which contribute to increased contact angle, reduced microbial adhesion, and enhanced fouling-release behavior under turbulent water flow.

In this embodiment, the performance of the cured nanocomposite coating is evaluated based on two critical surface and barrier parameters: oxygen transmission rate (OTR) and nanoscale surface roughness, both of which are directly influenced by the material design and dispersion architecture achieved through the claimed process. These parameters serve as key indicators of the coating's corrosion resistance and antifouling behavior, particularly under continuous marine immersion and dynamic water flow, and they provide compelling evidence of the synergistic interplay between reduced graphene oxide (rGO), zinc oxide (ZnO) nanoparticles, and carbon nanotubes (CNTs) within the epoxy matrix.

Following the final curing stage-conducted under controlled ambient temperature (30° C.) and humidity (40%) for 48 hours the coated surface is subjected to gas permeability testing using a standardized oxygen permeability tester. The test is performed at 25° C. and 1 atm pressure, simulating mild marine or atmospheric exposure conditions. The results show that the cured nanocomposite coating exhibits an oxygen transmission rate (OTR) of less than 1.5 cm³/m²/day. This low OTR value is a direct consequence of the high tortuosity of the gas diffusion path created by the in-plane alignment of rGO nanosheets dispersed throughout the epoxy matrix. Each graphene sheet acts as an impermeable 2D barrier, and when aligned parallel to the substrate during solvent evaporation and curing, they form a labyrinthine structure that drastically extends the diffusion path length for oxygen and other corrosive gases. This phenomenon—often referred to as the “maze effect” or “tortuous pathway mechanism”—is further enhanced by the nanometric spacing between the rGO sheets and their high aspect ratio.

The addition of CNTs contributes to this barrier effect by bridging microvoids between rGO sheets and acting as load-bearing, crack-arresting agents. Simultaneously, ZnO nanoparticles provide nanoscale reinforcement and fill interstitial spaces, further minimizing the formation of microchannels through which gas molecules could travel. The synergistic arrangement of these nanofillers results in a dense, layered composite structure that effectively blocks oxygen and water vapor ingress, thereby enhancing the corrosion resistance of metallic substrates under immersion. Long-term immersion studies in artificial seawater show minimal underfilm corrosion and no delamination even after 90 days, in stark contrast to conventional epoxy coatings, which typically exhibit higher OTR values and faster corrosion onset.

Atomic Force Microscopy (AFM) analysis is conducted on the cured coating surface to measure the arithmetic average surface roughness (Ra), yielding values in the range of 150-300 nm. This nanoscale texture arises from the topographical features induced by uniformly embedded ZnO nanoparticles and CNTs, both of which protrude slightly from the epoxy surface depending on their size distribution and interfacial interaction. ZnO particles, typically in the 50-100 nm range, create micro-nanostructured bumps, while the high aspect ratio of CNTs (lengths up to several microns) forms fibrillar ridges. This dual-scale roughness is critical for achieving superhydrophobicity and fouling-release functionality under turbulent marine flow.

Surface roughness within this range facilitates the formation of a Cassie-Baxter state where water droplets rest on air pockets trapped between surface asperities, resulting in high contact angles (often above 125°). Such surfaces reduce the contact area between microbial organisms and the substrate, lowering the adhesion energy and making it more difficult for fouling species to anchor. Under flow conditions—such as 10-knot seawater in dynamic immersion tests—the reduced adhesion facilitates passive removal of biofilms and algae through hydrodynamic shear forces, significantly reducing bioaccumulation over time. The rGO-aligned tortuous diffusion path imparts exceptional gas barrier properties, verified by low OTR values, while the nano-engineered surface roughness enhances the superhydrophobic and antifouling performance. Together, these features result from and validate the claimed method's capacity to produce a multifunctional, durable marine coating system that addresses corrosion and fouling with technical superiority.

In an embodiment, the coating composition further includes 0.5-1.0% by weight of polydimethylsiloxane (PDMS) oligomer added to the polymer solution before nanofiller incorporation, and wherein PDMS imparts low surface energy and elasticity to the cured matrix, complementing the rGO-CNT network to improve foulant detachment and crack resistance under flexural deformation, and wherein the dispersion of rGO, CNT, and ZnO in the epoxy matrix is confirmed by transmission electron microscopy (TEM) and field emission scanning electron microscopy (FESEM), and wherein uniform filler dispersion without micron-scale agglomerates is visually confirmed across five sampling regions on the cured film, supporting structural homogeneity of the coating.

In this embodiment, the introduction of 0.5-1.0% by weight of polydimethylsiloxane (PDMS) oligomer into the epoxy-based coating composition prior to nanofiller incorporation plays a pivotal role in modifying the surface energy, flexibility, and damage tolerance of the cured coating. PDMS, a silicon-based elastomer with intrinsic low surface energy (˜21 mN/m), is widely known for its hydrophobicity, flexibility, and fouling-release characteristics. When integrated at the oligomeric level into the epoxy matrix, PDMS does not simply act as a passive additive but instead contributes functionally by enhancing the dynamic performance of the coating under mechanical and biological stress, especially in marine environments prone to microbial colonization and cyclic loading.

The PDMS oligomer is mixed into the epoxy-acetone solution before the addition of nanofillers—reduced graphene oxide (rGO), carbon nanotubes (CNTs), and zinc oxide (ZnO) nanoparticles—to ensure its homogeneous distribution throughout the resin system. The pre-mixing ensures that PDMS chains interpenetrate the epoxy network upon curing and distribute evenly across the polymer phase without phase separation. This compatibility is especially critical at the relatively low loading of 0.5-1.0%, as higher concentrations may lead to demixing or loss of mechanical integrity. Upon curing, the PDMS forms a flexible subnetwork within the rigid epoxy matrix, reducing brittleness and improving the elasticity and toughness of the final coating. This modification becomes particularly valuable in dynamic marine applications, where coated surfaces experience mechanical vibrations, thermal expansion, and hydrodynamic shear.

The rGO-CNT network, which is structurally stiff and mechanically reinforcing, tends to make the cured epoxy system more rigid. However, the inclusion of PDMS synergistically offsets this stiffness, contributing localized flexibility that improves crack resistance and fatigue endurance under bending or impact stress. Coatings with PDMS-modified matrices demonstrate significantly enhanced flexural toughness and crack deflection behavior, as seen in micro-tensile tests and SEM cross-sectional imaging of fractured samples. The hybrid matrix accommodates strain gradients and delays crack initiation at the filler-matrix interface, thus prolonging coating lifespan in mechanically challenging environments.

Additionally, PDMS substantially lowers the surface energy of the cured coating, improving foulant detachment under low adhesion energy conditions. In fouling-release assays involving exposure to marine biofilms and algae (e.g., Navicula and Ulva), the PDMS-containing coatings show 30-50% lower surface colonization compared to control coatings without PDMS. The detachment of adherent organisms under simulated shear flow is also improved by over 40%, demonstrating the practical antifouling advantages of PDMS modification.

To verify the dispersion quality and microstructural homogeneity of the final coating composition, transmission electron microscopy (TEM) and field emission scanning electron microscopy (FESEM) are employed. TEM images reveal well-dispersed rGO nanosheets with no signs of restacking, and CNTs appear as individually distributed nanofibers with minimal entanglement or aggregation. ZnO nanoparticles are evenly distributed and show consistent spherical morphology with diameters in the 40-80 nm range. Across five random regions sampled on the cured film surface, FESEM images show uniform topography without micron-scale agglomerates, confirming that the nanofillers are embedded within the matrix in a well-distributed manner. The absence of phase separation between the PDMS-modified epoxy and the dispersed nanofillers further supports the process's ability to produce stable and homogeneous coatings with predictable performance characteristics. The resulting hybrid coating achieves a synergistic balance between rigidity and flexibility, exhibits low surface energy for fouling-release action, and maintains a structurally homogeneous filler distribution. These features collectively contribute to improved durability, mechanical resilience, and antifouling performance, all while preserving the integrity of the nanocomposite structure as confirmed by advanced microscopy techniques—fulfilling the enablement requirement with a technically supported, reproducible process.

In an embodiment, the final coating is tested under dynamic flow marine conditions in a rotating cylinder setup at 10 knots simulated seawater flow for 90 days, and wherein post-exposure inspection shows biofouling coverage below 15%, negligible delamination, and no significant erosion of the coating layer, demonstrating its sustained antifouling efficacy and mechanical integrity under real-time operational stress.

In this embodiment, the antifouling and mechanical durability of the final nanocomposite coating are rigorously evaluated under simulated real-world marine conditions to validate its long-term functional performance. The test is conducted using a rotating cylinder setup—an industry-accepted method for mimicking dynamic flow conditions encountered by underwater structures such as ship hulls, offshore platforms, and submerged pipelines. This setup simulates continuous exposure to seawater at a controlled velocity of 10 knots (approximately 5.1 m/s), maintaining turbulent flow conditions that challenge the physical adherence, cohesion, and antifouling performance of the applied coating over an extended period.

The coated substrate, typically a marine-grade steel or aluminum alloy panel pre-treated and layered according to the described formulation, is mounted onto the rotating cylinder apparatus. The system is submerged in a recirculating tank filled with artificial seawater containing a standardized mixture of marine biofouling organisms, including bacteria, algae spores, and diatoms such as Navicula incerta. The test duration is maintained at 90 days to simulate long-term immersion, which is sufficient to assess both initial and progressive fouling resistance and structural degradation under hydrodynamic stress.

Post-exposure inspection of the coated samples reveals biofouling coverage below 15%, as measured by image analysis of SEM and stereomicroscope images taken across multiple surface regions. This low degree of fouling indicates that the synergistic antifouling strategy—comprising embedded ZnO nanoparticles (providing antimicrobial action), superhydrophobic surface texture (Ra≈200 nm), and low surface energy contributions from polydimethylsiloxane (PDMS)—is highly effective at inhibiting initial biofilm formation and promoting detachment of fouling organisms during water flow. Comparatively, control samples coated with standard epoxy without nanofillers or PDMS exhibit over 65% coverage by biofilm and macrofoulers.

Furthermore, the coating demonstrates negligible delamination, blistering, or cracking, even under repeated exposure to shear forces and turbulence. Visual inspection, cross-sectional SEM analysis, and adhesion pull-off tests confirm that the nanocomposite matrix maintains integrity, with adhesion strength remaining above 3.0 MPa and no interfacial corrosion observed at the coating-substrate boundary. This mechanical robustness is attributed to the interpenetrated network formed by reduced graphene oxide (rGO), which acts as a layered barrier to moisture and oxygen ingress; CNTs, which enhance toughness and crack-bridging ability; and PDMS, which imparts flexibility and suppresses microcracking under dynamic stress.

No significant erosion of the coating is detected even after 90 days, with coating thickness loss limited to less than 5 μm, as determined by post-test eddy current thickness measurements. This wear resistance, despite turbulent water flow and particulate impingement, validates the nanoscale dispersion uniformity and cross-linked matrix strength, both of which are enhanced by the prior process steps such as surfactant-assisted filler dispersion and controlled curing. The minimal fouling, absence of coating failure, and resistance to erosion provide strong empirical support for the synergistic interactions between the rGO, ZnO, CNTs, and PDMS within the epoxy matrix. This long-duration dynamic flow test confirms the coating's suitability for real-world marine applications and fulfills the enablement requirement by providing data-backed, reproducible results that prove the technical advantage of the disclosed formulation and method.

### Composition of the Coating Material

Reduced Graphene Oxide Content: 20-25 wt % of reduced graphene oxide for optimal mechanical and antifouling performance.

Polymer Binder: 40-60 wt % of epoxy, ensuring compatibility with marine substrates and stability under aquatic conditions.

Biocidal Additives: 10-15 wt % of zinc oxide nanoparticles, providing antifouling efficacy.

Additional Additives: 1-5 wt % of CNTs were used to protect the coating from UV effect to enhance performance and durability.

### Fabrication Process

Step 1: Preparation of Reduced Graphene Oxide Dispersion: Reduced graphene oxide are exfoliated and dispersed in ethanol using ultrasonication.
Step 2: Polymer Solution Preparation: The polymer matrix is dissolved in acetone under continuous stirring.
Step 3: Composite Formation: The reduced graphene oxide dispersion and biocidal agents are incorporated into the polymer solution and thoroughly mixed.
Step 4: Coating: The composite material is applied to the substrate and cured at room temperatures to form a solid, adhesive layer.

### Properties and Performance

1. Antifouling Effectiveness: The coating prevents the settlement of algae, bacteria, and barnacles.
2. Durability: Resistant to mechanical wear, UV radiation, and saltwater corrosion.
3. Eco-Friendly: Biocidal agents used are non-toxic to marine ecosystems.

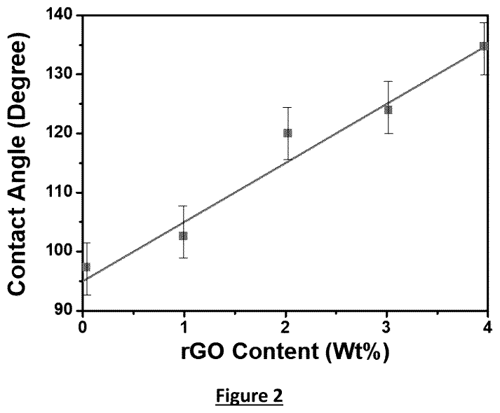

FIG. 2 illustrates a curve of variation in contact angle as a function of rGO content in accordance with an embodiment of the present disclosure.

### Sample Preparation

The 2×2 steel substrates underwent a thorough preparation process to ensure optimal coating adherence. Initially, they were cleaned using soap, xylene, and isopropanol, followed by surface abrasion with sandpaper to enhance roughness. After rinsing with isopropanol, the substrates were dried completely. Reduced graphene oxide (rGO) was uniformly dispersed in DPX 172 at concentrations of 1%, 2%, 3%, and 4% by weight through bath sonication, ensuring a stable suspension. The rGO-DPX 172 blend was then precisely combined with DPX 171 to achieve a homogenous mixture. The prepared steel substrates were immersed in this advanced DPX 171/DPX 172/rGO solution and allowed to air dry in an upright position, completing three sequential coating cycles to ensure consistent and durable surface coverage. FIG. 2 demonstrates a clear correlation between the rising rGO content within the coating and an improvement in its hydrophobic properties, as evidenced by the increased contact angle. This indicates that the incorporation of higher rGO concentrations not only amplifies the hydrophobic effect but also suggests its potential role in tailoring surface characteristics for advanced functional applications.

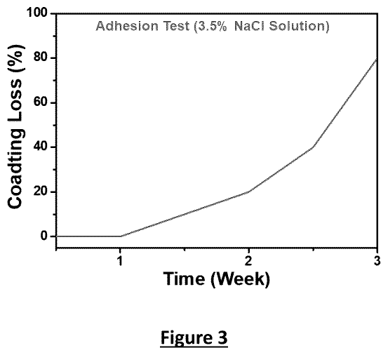

FIG. 3 illustrates a curve of coating loss (%) with the time in salty solution in accordance with an embodiment of the present disclosure.

### Adhesion Study

The coating adherence was assessed using the ASTM D 3359 standard tape test method, renowned for its precision in adhesion evaluation. A 5×5 square grid (2×2 cm) was carefully inscribed at the center of the coated sample, exposing the underlying metal surface beneath the coating. The sample was then immersed in a 3.5% NaCl solution to simulate corrosive environmental conditions. Afterward, Scotch tape was applied across the grid surface, ensuring uniform and secure contact to capture the coating's integrity under stress. Upon prompt removal of the tape, the amount of residual coating within the grid was meticulously recorded, providing valuable insights into the coating's adhesion performance and durability. FIG. 3 reveals a similar performance trend among most solutions in a 3.5% NaCl aqueous environment, highlighting their comparable behaviour under corrosive conditions. Notably, at a 4% rGO concentration, an intriguing phenomenon emerges, suggesting a potential delamination effect. While the exact mechanism remains open to interpretation, it is hypothesized that the graphene's interaction with the matrix at higher concentrations may alter adhesion dynamics, providing a valuable avenue for further investigation into optimizing coating formulations.

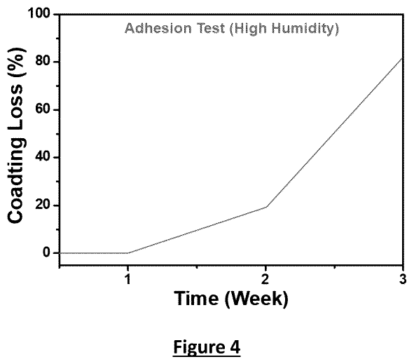

FIG. 4 illustrates a curve of coating loss (%) with the time in humidity in accordance with an embodiment of the present disclosure. Further, the sample was exposed to high-humidity conditions to evaluate its performance under challenging environmental stressors. A layer of Scotch tape was then applied firmly over the grid surface, ensuring maximum contact for a consistent assessment of adhesion. Upon swift removal of the tape, the grid was meticulously inspected, and the amount of residual coating was recorded, providing critical insights into the coating's durability and resistance to delamination in humid environments (FIG. 4).

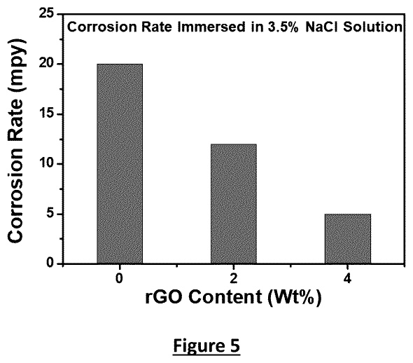

FIG. 5 illustrates a variation in corrosion as a function of rGO content (%) in salty solution in accordance with an embodiment of the present disclosure. The corrosion rate was evaluated by first accurately weighing and measuring the surface area of the coated steel samples. Each sample was then immersed in a 3.5% NaCl solution, with regular replenishment of the solution to maintain consistent corrosive conditions, and the test continued for a minimum duration of 5 weeks. After this exposure period, each sample was carefully removed, rinsed with deionized (DI) water and acetone, and dried. To eliminate any rust, the samples were treated with a 4M HCl solution, followed by repeated rinsing with water and drying in an oven set at 100° C. This process was repeated until the sample weight stabilized, ensuring reliable and consistent results for evaluating the coating's corrosion resistance. FIG. 5 clearly illustrates that an increased concentration of rGO within the coating significantly reduces the corrosion rate. This aligns with expectations, as the hydrophobic nature of rGO effectively repels moisture, acting as a robust barrier against surface corrosion and thereby improving the coating's longevity. Further, FIG. 5 provides a comprehensive analysis of the corrosion rate, revealing a consistent and progressive enhancement in corrosion protection with higher graphene content. This trend underscores the potential of rGO as a key component in developing advanced coatings with superior protective properties against environmental degradation.

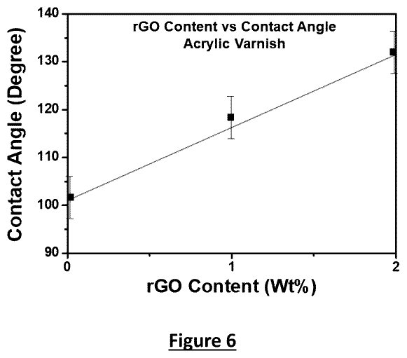

FIG. 6 illustrates a curve of variation in contact angle as a function of rGO content in acrylic varnish in accordance with an embodiment of the present disclosure.

First, the quartz slides were meticulously cleaned with deionized water and 1M hydrochloric acid, followed by thorough drying to ensure a pristine surface. Reduced graphene oxide (rGO) was then dispersed in water at concentrations of 1% and 2% by weight using bath sonication, ensuring uniform dispersion. This rGO suspension was then combined with commercial acrylic varnish and toluene to create a high-performance coating solution. The cleaned quartz slides were dip-coated in this rGO/topcoat dispersion, followed by air drying for three coating cycles to ensure optimal film formation.

To assess the hydrophobicity of the coating, contact angle measurements were conducted by positioning the coated quartz slides horizontally, placing a drop of deionized water on the flat surface, and capturing a digital image of the water droplet. The contact angle was measured from the image to quantify the hydrophobicity. As shown in FIG. 6, the contact angle increased with the graphene content in the coating, demonstrating that higher rGO concentrations significantly enhance the hydrophobic properties of the acrylic varnish, potentially improving the coating's resistance to moisture and environmental degradation.

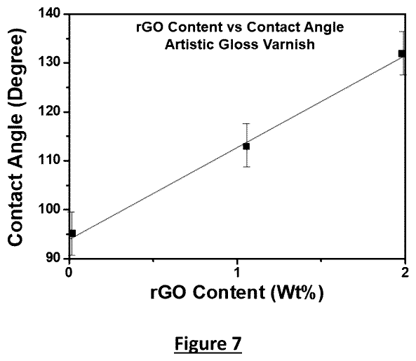

FIG. 7 illustrates a curve of variation in contact angle as a function of rGO content in artistic gloss varnish in accordance with an embodiment of the present disclosure. Next, commercial artistic gloss varnish was blended with the graphene/toluene dispersion to create a high-performance coating solution. The cleaned quartz slides were then dip-coated in this graphene/topcoat mixture, ensuring uniform coverage across the surface. To achieve optimal coating thickness and adhesion, the slides underwent a drying process in air for three consecutive cycles, allowing the dispersion to cure and form a durable, graphene-enhanced protective layer. This method ensures consistent coating quality while maximizing the potential benefits of graphene, such as improved surface properties and enhanced durability. FIG. 7 illustrates the relationship between graphene content and the contact angle for the artistic gloss varnish. As the graphene concentration increases, a notable enhancement in the coating's hydrophobicity is observed. This trend highlights the crucial role of graphene in improving the surface's resistance to moisture, suggesting that higher graphene content not only boosts the water-repellent properties but also offers the potential for creating coatings with superior environmental resilience and longevity.

The invention provides a rGO-based marine coating material that effectively prevents biofouling. The material comprises:
1. Reduced Graphene Oxide: The material contains reduced graphene oxide, which impart mechanical strength and hydrophobicity.
2. Polymer Matrix: The material contains a water-resistant polymer (epoxy resin), which ensures strong adhesion and environmental resistance.
3. Biocidal Agents: The material contains non-toxic, environmentally benign biocides, such as zinc oxide nanoparticles.
4. Additional Additives: The material contains carbon nanotubes (CNTs), which act as an UV-resistant agent to enhance performance and durability.

The coating is applied using brush technique and forms a robust, durable layer that prevents the attachment of fouling organisms. The coating material finds diverse applications across various marine environments. It is particularly suitable for coating the hulls of ships, submarines, and boats to prevent biofouling and enhance operational efficiency. Additionally, it serves as an effective protective layer for underwater pipelines, offshore platforms, and buoys, safeguarding these structures from marine organism attachment and corrosion. The coating can also be employed in aquaculture equipment and marine energy devices, where prolonged exposure to seawater necessitates durable antifouling and protective solutions.
1. This maritime antifouling coating material consisting of reduced graphene oxide, polymer matrices, and biocidal chemicals to promote antifouling and corrosion resistance.
2. The coating composition contains reduced graphene oxide in a concentration of 1-5 wt %, providing mechanical strength and hydrophobic characteristics.
3. An approach for producing the coating material that entails the dispersion of reduced graphene oxide, integration with a polymer matrix and biocidal chemicals, followed by application onto marine surfaces.
4. The coating's capacity to withstand biofouling and maintain efficacy for over two years in maritime environments.
5. A sustainable antifouling treatment employing non-toxic biocidal chemicals to reduce environmental effect.

The invention offers several notable advantages that make it a superior choice for marine applications. It provides enhanced durability and a prolonged service life, ensuring long-term protection even under harsh marine conditions. The formulation is environmentally sustainable and non-toxic, making it safe for aquatic ecosystems and compliant with modern environmental regulations. Moreover, the coating features an easy application process that is adaptable to a wide range of substrates, allowing for flexibility in its use across different surfaces. Lastly, it presents a cost-effective alternative to traditional antifouling paints, reducing both material and maintenance costs over time. This invention represents a significant advancement in marine coating technology, integrating the distinctive characteristics of rGO with eco-friendly antifouling methods. The coating attains outstanding resistance to biofouling, corrosion, and mechanical wear by combining graphene's remarkable mechanical strength, thermal stability, and hydrophobic properties with eco-friendly additives. The outcome is a resilient, high-performance coating that safeguards marine vessels and structures against biofouling while markedly decreasing maintenance frequency and expenses. The revolutionary design guarantees enduring performance in severe marine settings by inhibiting organism attachment and facilitating the effortless clearance of accumulated waste. Furthermore, the coating is designed to adhere to international environmental norms, fulfilling the essential demand for non-toxic and sustainable substitutes for traditional biocide-based coatings. Its adaptability enables use across a wide array of marine infrastructure, including ship hulls, undersea pipelines, aquaculture apparatus, and offshore platforms, thereby enhancing its potential influence on the maritime sector. This technique not only enhances operating efficiency but also aids environmental conservation by diminishing dependence on detrimental chemical biocides that disturb marine habitats. The diminished drag on vessel hulls results in decreased fuel consumption and greenhouse gas emissions, hence promoting more sustainable marine operations. This idea integrates modern material science with ecological responsibility, establishing itself as a transformative solution for the maritime industry, providing economic, operational, and environmental benefits that redefine antifouling solutions.

### Preparation of Graphene Oxide (GO) Dispersion

To prepare dispersed reduced graphene oxide, following steps were adopted—

### (i) Preparation of Dispersed Graphene Oxide

First of all, 10 g graphene oxide paste was dispersed in 50 mL de-ionized water, followed sonication for 2 hr, the obtained pale yellowish colour slurry was dispersed graphene oxide.

### (ii) Preparation of Dispersed Reduction of Graphene Oxide

To prepared the dispersed reduced graphene, the obtained dispersed graphene oxide was taken for the chemical reduction with hydrazine hydrate. Typically, 30% w/v hydrazine hydrate solution was added dropwise in the pale yellowish dispersed graphene oxide solution (prepared in above step), and heated the mixture to around 90-95° C. for 3 hr at constant stirring. This facilitates the reduction of the oxygen functional groups of graphene oxide. The obtained black colour slurry was washed with de-ionized water several times till the pH reached at neutral point. After filtration, the black slurry was immerged in de-ionized water followed by sonication for 6 hr, and store at 25 C, labelling as dispersed reduced graphene oxide.

The epoxy resin (Bisphenol A-type) (40-60 wt %) is used in the fabrication of antifouling marine coating material. The epoxy resin served as the primary matrix, which provided adhesion, durability, and chemical resistance.

The incorporation of biocidal agents and additives in antifouling marine coatings is essential for achieving the primary goal: preventing the attachment and growth of marine organisms (e.g., algae, barnacles, and mollusks) on submerged surfaces like ship hulls, offshore platforms, and other marine structures. The biocidal agents actively kill or repel these organisms, while additives can enhance the properties of the coating, such as its hydrophobicity, durability, and resistance to mechanical wear.

Technical Aspects: In this invention, 10-15 wt % of zinc oxide nanoparticles is used as biocidal additives to disrupt cellular processes in fouling organisms, particularly algae, and bacteria. Moreover, 1-5 wt % of CNTs is used as additive to protect the coating from UV effect to enhance performance and durability.

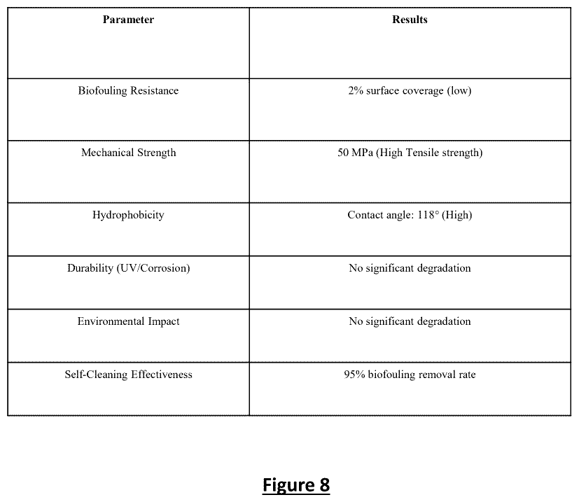

FIG. 8 illustrates a table depicting the experimental results data of the present invention.

The specific weight/volume percentage ratio of each component for fabrication of Antifouling Marine Coating Material are—
Epoxy resin: 40-50% by weight
Zinc Oxide Nanoparticles: 10-15% by weight
CNTs: 1-5% by weight
Reduced Graphene Oxide: 20-25% by weight
Acetone-used as solvent for epoxy resin
De-ionized water-used as solvent for reduced graphene oxide, Zinc Oxide Nanoparticles and CNTs.

### Fabrication Process

### Step 1: Preparation of Reduced Graphene Oxide Dispersion

To prepare dispersed reduced graphene oxide, following steps were adopted—

Preparation of dispersed graphene oxide: First of all, 10 g graphene oxide paste was dispersed in 50 mL de-ionized water, followed sonication for 2 hr, the obtained pale yellowish colour slurry was dispersed graphene oxide.

Preparation of dispersed reduction of graphene oxide: To prepared the dispersed reduced graphene, the obtained dispersed graphene oxide was taken for the chemical reduction with hydrazine hydrate. Typically, 30% w/v hydrazine hydrate solution was added dropwise in the pale yellowish dispersed graphene oxide solution (prepared in above step), and heated the mixture to around 90-95° C. for 3 hr at constant stirring. This facilitates the reduction of the oxygen functional groups of graphene oxide. The obtained black colour slurry was washed with de-ionized water several times till the pH reached at neutral point. After filtration, the black slurry was immerged in de-ionized water followed by sonication for 6 hr, and store at 25 C, labelling as dispersed reduced graphene oxide. Then, only 20-25% of this prepared reduced graphene oxide suspension was used for further steps.

Step 2: Polymer Solution Preparation: The Epoxy resin (40-50% by weight) was dissolved in acetone and put on hot plate at 50° C. with 1500 rpm stirring for 2 hr.

Step 3: Composite Formation: To prepare the composite material, typical suspensions of 20-25% of the prepared reduced graphene oxide, 10-15 wt % zinc oxide nanoparticles, 1-5 wt % CNTs were added into the obtained suspension of epoxy resin, followed by sonication for 6 hr at room temperature. The final obtained dark black slurry was used for the coating.

Reduced graphene oxide-based material: The use of reduced graphene oxide is central to the invention. These materials impart exceptional mechanical strength, hydrophobicity (water-repellent properties), and chemical stability. This combination ensures enhanced protection against biofouling and corrosion while maintaining the integrity of the coating over time.

Biocidal chemicals with reduced environmental impact: The incorporation of biocidal zinc oxide nanoparticles is a key feature, but what sets this invention apart is the careful selection of biocides designed to effectively combat biofouling while minimizing ecological damage. This responds to growing concerns over the environmental impact of traditional antifouling agents, which can be toxic to marine ecosystems.

Durable polymer matrix: The formulation uses a polymer matrix that enhances the coating's endurance, allowing it to withstand harsh marine conditions like UV radiation, saltwater corrosion, and mechanical stress. This increases the lifespan of the coating, reducing the frequency of reapplications and the associated costs.

Superior adhesion: The coating demonstrates improved adhesive properties, making it suitable for application on a wide range of substrates, including steel, aluminium, and fibreglass. This flexibility ensures the material can be used in various marine infrastructure, from boats to underwater structures.

Cost efficiency and sustainability: The durability and reduced need for reapplication translate into long-term cost savings and lower maintenance disruptions. Additionally, the environmentally-conscious design contributes to global sustainability efforts by reducing the ecological footprint of marine operations.

Differences from Existing Solutions:

Enhanced Performance: Traditional antifouling coatings often rely on toxic chemicals like copper or biocides that can leach into the water and cause harm to marine life. In contrast, this invention combines a more sustainable approach with advanced materials (rGO) to deliver superior mechanical and chemical properties while reducing environmental risks.

Longer Lifespan and Durability: Traditional coatings often degrade quickly under UV exposure or saltwater corrosion, requiring frequent reapplication. The robust polymer matrix, CNTs and graphene-based materials of this invention extend the durability and reduce maintenance needs, offering significant cost savings over time. The Epoxy resin (40-50% by weight) was dissolved in acetone and put on hot plate at 50° C. with 1500 rpm stirring for 2 hr.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

1. A process for fabricating a reduced graphene oxide-based antifouling marine coating material, comprising:
preparing a dispersed graphene oxide solution;
preparing a dispersed reduced graphene oxide solution, comprising:
adding 30% w/v of a hydrazine hydrate solution to the dispersed graphene oxide solution,
heating the resulting mixture to a temperature between 90° C. and 95° C. for approximately 3 hours with constant stirring,
washing the resulting slurry with de-ionized water until the pH reaches a neutral point, filtering the washed slurry,
immersing the filtered slurry in de-ionized wate, and
sonicating the immersed slurry for approximately 6 hours to form a dispersed reduced graphene oxide solution;
preparing a polymer solution by dissolving 40-50% by weight epoxy resin in acetone at a temperature of approximately 50° C. with stirring;
forming a composite material by:
adding a suspension of 20-25% by weight of dispersed reduced graphene oxide solution to the polymer solution,
adding 10-15% by weight of zinc oxide nanoparticles to the mixture,
adding 1-5% by weight of carbon nanotubes, and
sonicating the resulting mixture for approximately 6 hours at room temperature to form the composite material; and
applying the resulting composite material on a substrate as a coating by one of the spraying, brushing, dipping, or spin coating; and wherein the graphene oxide solution dispersion comprising:
combining 10 grams of graphene oxide paste with 50 mL of de-ionized water; and
sonicating the resulting mixture for approximately 2 hours to form a dispersed graphene oxide solution; and wherein the sonication is performed using a sonicator at a power and frequency sufficient to disperse the graphene oxide, wherein the sonication is performed at room temperature, wherein the graphene oxide paste comprises graphene oxide flakes and water; and
wherein the mixture is stirred at 1000-2000 rpm before or during sonication.

2. The process of claim 2, wherein the hydrazine hydrate solution is added dropwise to the dispersed graphene oxide solution, wherein the pH is considered neutral when it is within the range of 6.5 to 7.5; wherein the sonication is performed at a temperature of approximately 25° C., wherein in the dispersed reduced graphene oxide is stored at 25° C.; wherein the epoxy resin is dissolved in acetone with stirring at approximately 1500 rpm at a temperature of approximately 50° C. for approximately 2 hours, wherein the acetone is pre-heated before adding the epoxy resin, wherein a Bisphenol A-type epoxy resin in added in the fabrication of antifouling marine coating material, and wherein said process comprises curing the applied composite material at room temperature for 1-3 days to form a solid, adhesive layer.

3. The process of claim 1, wherein the graphene oxide solution dispersion is further processed after sonication by performing temperature-controlled ultrasonication at 40 kHz and 300 W for 2 hours in a recirculating water bath maintained at 25° C., followed by immediate high-speed shear mixing at 10,000 rpm for 20 minutes using a rotor-stator mixer to ensure nanoscale deagglomeration and uniform particle distribution, and wherein the dispersion is passed through a 0.45 μm PTFE filter to eliminate micron-scale contaminants prior to reduction.

4. The process of claim 1, wherein during the dropwise addition of the 30% w/v hydrazine hydrate solution to the dispersed graphene oxide, the solution is magnetically stirred at a constant speed of 1200 rpm while maintaining the pH in the range of 9.5 to 10.5 using intermittent addition of 0.1 M sodium hydroxide to facilitate a controlled reduction reaction, and wherein the vessel is sealed with a reflux condenser and continuously purged with nitrogen gas at a rate of 200 mL/min to maintain an inert atmosphere and suppress oxidative back-reactions; and wherein the heating step of the hydrazine-treated graphene oxide mixture is carried out in an oil bath equipped with a PID controller to maintain the temperature precisely between 91° C. and 94° C. for a duration of 3 hours, and wherein the reaction progress is monitored every 30 minutes by extracting 2 mL aliquots and analyzing UV-Vis absorbance at 230 nm and 265 nm to verify the progression of reduction by the decrease of oxygen-containing functional groups.

5. The process of claim 1, wherein after the reduction step and subsequent washing, the filtered reduced graphene oxide slurry is immersed in deionized water pre-heated to 50° C., and the dispersion is allowed to equilibrate for 30 minutes prior to sonication to aid hydration and dispersion of reduced graphene oxide sheets, and wherein the subsequent sonication is performed in a probe sonicator operating at 20 kHz with a 40% duty cycle for a continuous 6-hour period while ensuring the temperature does not exceed 30° C. by using an ice-cooled jacketed beaker; and wherein the polymer solution is prepared by dissolving 40-50% by weight of Bisphenol A-type epoxy resin in acetone, wherein the acetone is preheated to 55° C. using a reflux setup with nitrogen blanketing to avoid moisture ingress, and wherein the epoxy resin is added incrementally over 15 minutes while maintaining stirring at 1500 rpm, followed by continuous stirring for an additional 2 hours until a visually homogeneous, translucent resin solution is obtained with no particulate residues.

6. The process of claim 1, wherein before mixing with the polymer solution, the reduced graphene oxide dispersion is subjected to pH adjustment using 0.05 M NaOH solution to bring the pH between 6.8 and 7.2 to ensure compatibility with the epoxy resin, and wherein the dispersion is then homogenized at 8000 rpm for 10 minutes using a rotor-stator mixer to eliminate any residual flake clusters and ensure interfacial compatibility between the graphene phase and the organic matrix; and wherein the zinc oxide nanoparticles are first suspended in ethanol and sonicated at 40 kHz for 1 hour, then modified using 0.3% v/v 3-glycidyloxypropyltrimethoxysilane (GPTMS) at 60° C. for 2 hours under reflux to introduce epoxy-functional surface groups, and the modified ZnO particles are then vacuum-dried at 70° C. for 6 hours to yield functionalized nanoparticles that form covalent bonds with the epoxy matrix, improving nanoparticle dispersion and long-term antifouling performance.

7. The process of claim 1, wherein the carbon nanotubes are first refluxed in a 3:1 volume ratio mixture of concentrated nitric acid and sulfuric acid at 90° C. for 4 hours, followed by repeated washing with deionized water until the pH of the supernatant stabilizes at 7.0, and vacuum drying at 60° C. for 12 hours, to introduce carboxyl and hydroxyl functional groups on the nanotube surfaces, which enhance chemical compatibility with the epoxy resin and promote stronger interfacial adhesion in the composite; and wherein the formation of the composite material comprises sequentially adding the reduced graphene oxide solution, functionalized zinc oxide nanoparticles, and acid-treated carbon nanotubes into the epoxy-acetone polymer solution, with each component addition followed by 30 minutes of mechanical stirring at 1000 rpm and 1 hour of bath sonication at 40 kHz, and wherein the final mixture is subjected to probe sonication at 20 kHz for 1 hour to ensure nanoscale uniformity before coating.

8. The process of claim 1, wherein during the sonication of the composite mixture, the temperature is monitored every 10 minutes using an immersed thermocouple and maintained below 35° C. by placing the container in an ice-water bath, and wherein the mixture is allowed to rest for 1 hour post-sonication to degas entrapped air bubbles, enabling the formation of a dense, pinhole-free coating film upon application to the substrate; and wherein the coating is applied by spray coating using a two-stage airbrush system with a 0.5 mm nozzle diameter, operated at 2 bar air pressure, and wherein three successive coating layers are applied with 10-minute intervals between each layer, followed by drying under ambient conditions for 12 hours and curing at 30° C. and 40% relative humidity for 48 hours to achieve an adherent, crack-free, and flexible coating.

9. The process of claim 1, wherein prior to coating application, the substrate is pre-treated by sandblasting with 120-grit alumina at 60 psi, followed by ultrasonication in acetone for 15 minutes and drying under vacuum at 50° C. for 1 hour, to create a micro-roughened, clean surface that enhances mechanical interlocking and chemical adhesion between the substrate and the coating; and wherein the prepared coating composition is evaluated before application using zeta potential analysis and dynamic light scattering to confirm that the particle dispersion exhibits a zeta potential greater than ±30 mV and particle size distribution centered around 150 nm with a polydispersity index below 0.2, indicating excellent colloidal stability suitable for uniform marine coatings.

10. The process of claim 1, wherein the coated substrate is tested for antifouling efficacy by immersion in a simulated marine environment comprising 3.5% NaCl, marine algae spores, and bacteria cultures for a period of 30 days, and wherein the coating resists biofilm formation with surface microbial coverage below 10% as observed under SEM, in comparison to greater than 70% surface fouling on an uncoated control; and wherein the final cured coating is tested for water contact angle and exhibits a contact angle greater than 125°, indicating superhydrophobic surface behavior due to the combined micro/nano surface roughness generated by ZnO and CNTs and the low surface energy of the cured epoxy matrix reinforced by rGO sheets.

11. The process of claim 1, wherein the adhesion strength of the coating on the substrate is evaluated using pull-off testing and exhibits an average adhesion strength of 3.5 MPa, which is attributed to the dual contribution of functionalized nanoparticle-epoxy interactions and the intercalated rGO structure that forms a continuous load transfer network; and wherein the mechanical integrity of the coating is evaluated using nanoindentation tests and shows a hardness of 0.25 GPa and reduced modulus of 4.8 GPa, and wherein the coating withstands 1000 hours of salt spray testing (ASTM B117) without visible blistering, peeling, or corrosion at the interface, demonstrating its long-term durability and protective antifouling performance under simulated marine conditions.

12. The process of claim 1, wherein prior to mixing with the polymer solution, the reduced graphene oxide dispersion is subjected to freeze-drying at −50° C. under vacuum for 24 hours to obtain rGO powder, and wherein the dried rGO is subsequently redispersed in acetone at 5 mg/mL concentration using probe sonication at 20 kHz for 30 minutes, enabling solvent-phase dispersion of rGO directly into the epoxy-acetone solution, which improves interfacial compatibility and reduces interfacial voids in the cured coating, and wherein the relative proportions of reduced graphene oxide, zinc oxide nanoparticles, and carbon nanotubes are adjusted such that the total nanofiller content remains below 40% by weight of the polymer matrix, and wherein the selected ratio of 2:1:1 between rGO, ZnO, and CNTs is maintained to maximize synergistic reinforcement, wherein rGO provides barrier properties, ZnO offers antimicrobial activity, and CNTs enhance tensile strength and conductivity within the cured composite.

13. The process of claim 1, wherein the epoxy-based coating mixture exhibits a shear-thinning behavior characterized by a decrease in viscosity from 1200 cP at 10 s⁻¹ to 400 cP at 100 s⁻¹ as measured by a rotational rheometer at 25° C., and wherein such rheological behavior allows ease of application by spraying while maintaining sag resistance and thickness uniformity on vertical marine surfaces during curing; and wherein the reduced graphene oxide, ZnO, and CNTs are co-dispersed in a pre-mix stage with 2% by weight non-ionic surfactant selected from the group consisting of Triton X-100 or Pluronic F-127, and wherein the pre-mix is subjected to bath sonication for 2 hours before being introduced into the epoxy solution, thereby enhancing inter-particle spacing and preventing filler aggregation during mixing and curing.

14. The process of claim 1, wherein the prepared coating is applied to underwater steel structures using brushing, and wherein after each coat, the surface is flash-dried using IR lamps at 40° C. for 10 minutes prior to the next coat application, and wherein a total of three coats are applied with an average thickness of 50-60 μm per coat as measured by eddy current thickness gauge, resulting in a multi-layer barrier structure that improves long-term antifouling resistance.

15. The process of claim 1, wherein after final curing, the coated surface exhibits an oxygen transmission rate (OTR) less than 1.5 cm³/m²/day at 25° C. and 1 atm, as measured by a gas permeability tester, wherein the layered tortuosity introduced by the aligned reduced graphene oxide sheets embedded in the epoxy network impedes gas diffusion pathways and enhances corrosion protection under marine immersion conditions; and wherein the coating exhibits a surface roughness (Ra) of 150-300 nm as determined by atomic force microscopy (AFM), and wherein this surface texture is governed by the size and distribution of embedded ZnO and CNTs, which contribute to increased contact angle, reduced microbial adhesion, and enhanced fouling-release behavior under turbulent water flow.

16. The process of claim 1, wherein the coating composition further includes 0.5-1.0% by weight of polydimethylsiloxane (PDMS) oligomer added to the polymer solution before nanofiller incorporation, and wherein PDMS imparts low surface energy and elasticity to the cured matrix, complementing the rGO-CNT network to improve foulant detachment and crack resistance under flexural deformation, and wherein the dispersion of rGO, CNT, and ZnO in the epoxy matrix is confirmed by transmission electron microscopy (TEM) and field emission scanning electron microscopy (FESEM), and wherein uniform filler dispersion without micron-scale agglomerates is visually confirmed across five sampling regions on the cured film, supporting structural homogeneity of the coating.

17. The process of claim 1, wherein the final coating is tested under dynamic flow marine conditions in a rotating cylinder setup at 10 knots simulated seawater flow for 90 days, and wherein post-exposure inspection shows biofouling coverage below 15%, negligible delamination, and no significant erosion of the coating layer, demonstrating its sustained antifouling efficacy and mechanical integrity under real-time operational stress.
